SEQUENCE LISTING (213 sequences)

SEQ ID NO: 1 - DNA, 1937 bp, Homo sapiens
gcactttcactctccgtcagccgcattgcccgctcggcgtccggcccccgacccgcgctcgtccgcccgcccgcccgcccgcccgcgccatgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagtaagcacaacagccaaaaaggactttccgctagacccactcgaggaaaactaaaaccttgtgagagatgaaagggcaaagacgtgggggagggggccttaaccatgaggaccaggtgtgtgtgtggggtgggcacattgatctgggatcgggcctgaggtttgccagcatttagaccctgcatttatagcatacggtatgatattgcagcttatattcatccatgccctgtacctgtgcacgttggaacttttattactggggtttttctaagaaagaaattgtattatcaacagcattttcaagcagttagttccttcatgatcatcacaatcatcatcattctcattctcattttttaaatcaacgagtacttcaagatctgaatttggcttgtttggagcatctcctctgctcccctggggagtctgggcacagtcaggtggtggcttaacagggagctggaaaaagtgtcctttcttcagacactgaggctcccgcagcagcgcccctcccaagaggaaggcctctgtggcactcagataccgactggggctgggcgccgccactgccttcacctcctctttcaacctcagtgattggctctgtgggctccatgtagaagccactattactgggactgtgctcagagacccctctcccagctattcctactctctccccgactccgagagcatgcttaatcttgcttctgcttctcatttctgtagcctgatcagcgccgcaccagccgggaagagggtgattgctggggctcgtgccctgcatccctctcctcccagggcctgccccacagctcgggccctctgtgagatccgtctttggcctcctccagaatggagctggccctctcctggggatgtgtaatggtccccctgcttacccgcaaaagacaagtctttacagaatcaaatgcaattttaaatctgagagctcgctttgagtgactgggttttgtgattgcctctgaagcctatgtatgccatggaggcactaacaaactctgaggtttccgaaatcagaagcgaaaaaatcagtgaataaaccatcatcttgccactaccccctcctgaagccacagcagggtttcaggttccaatcagaactgttggcaaggtgacatttccatgcataaatgcgatccacagaaggtcctggtggtatttgtaactttttgcaaggcatttttttatatatatttttgtgcacatttttttttacgtttctttagaaaacaaatgtatttcaaaatatatttatagtcgaacaattcatatatttgaagtggagccatatgaatgtcagtagtttatacttctctattatctcaaactactggcaatttgtaaagaaatatatatgatatataaatgtgattgcagcttttcaatgttagccacagtgtattttttcacttgtactaaaattgtatcaaatgtgacattatatgcactagcaataaaatgctaattgtttcatggtataaacgtcctactgtatgtgggaatttatttacctgaaataaaattcattagttgttagtgatggagcttaaaaaaaa

SEQ ID NO: 2 - DNA, 3542 bp, Homo sapiens
gcactttcactctccgtcagccgcattgcccgctcggcgtccggcccccgacccgcgctcgtccgcccgcccgcccgcccgcccgcgccatgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagaggttcaagatgtgagagggtcagacgcctgaggaacccttacagtaggagcccagctctgaaaccagtgttagggaagggcctgccacagcctcccctgccagggcagggccccaggcattgccaagggctttgttttgcacactttgccatattttcaccatttgattatgtagcaaaatacatgacatttatttttcatttagtttgattattcagtgtcactggcgacacgtagcagcttagactaaggccattattgtacttgccttattagagtgtctttccacggagccactcctctgactcagggctcctgggttttgtattctctgagctgtgcaggtggggagactgggctgagggagcctggccccatggtcagccctagggtggagagccaccaagagggacgcctgggggtgccaggaccagtcaacctgggcaaagcctagtgaaggcttctctctgtgggatgggatggtggagggccacatgggaggctcacccccttctccatccacatgggagccgggtctgcctcttctgggagggcagcagggctaccctgagctgaggcagcagtgtgaggccagggcagagtgagacccagccctcatcccgagcacctccacatcctccacgttctgctcatcattctctgtctcatccatcatcatgtgtgtccacgactgtctccatggccccgcaaaaggactctcaggaccaaagctttcatgtaaactgtgcaccaagcaggaaatgaaaatgtcttgtgttacctgaaaacactgtgcacatctgtgtcttgtttggaatattgtccattgtccaatcctatgtttttgttcaaagccagcgtcctcctctgtgaccaatgtcttgatgcatgcactgttccccctgtgcagccgctgagcgaggagatgctccttgggccctttgagtgcagtcctgatcagagccgtggtcctttggggtgaactaccttggttcccccactgatcacaaaaacatggtgggtccatgggcagagcccaagggaattcggtgtgcaccagggttgaccccagaggattgctgccccatcagtgctccctcacatgtcagtaccttcaaactagggccaagcccagcactgcttgaggaaaacaagcattcacaacttgtttttggtttttaaaacccagtccacaaaataaccaatcctggacatgaagattctttcccaattcacatctaacctcatcttcttcaccatttggcaatgccatcatctcctgccttcctcctgggccctctctgctctgcgtgtcacctgtgcttcgggcccttcccacaggacatttctctaagagaacaatgtgctatgtgaagagtaagtcaacctgcctgacatttggagtgttccccttccactgagggcagtcgatagagctgtattaagccacttaaaatgttcacttttgacaaaggcaagcacttgtgggtttttgttttgtttttcattcagtcttacgaatacttttgccctttgattaaagactccagttaaaaaaaattttaatgaagaaagtggaaaacaaggaagtcaaagcaaggaaactatgtaacatgtaggaagtaggaagtaaattatagtgatgtaatcttgaattgtaactgttcttgaatttaataatctgtagggtaattagtaacatgtgttaagtattttcataagtatttcaaattggagcttcatggcagaaggcaaacccatcaacaaaaattgtcccttaaacaaaaattaaaatcctcaatccagctatgttatattgaaaaaatagagcctgagggatctttactagttataaagatacagaactctttcaaaaccttttgaaattaacctctcactataccagtataattgagttttcagtggggcagtcattatccaggtaatccaagatattttaaaatctgtcacgtagaacttggatgtacctgcccccaatccatgaaccaagaccattgaattcttggttgaggaaacaaacatgaccctaaatcttgactacagtcaggaaaggaatcatttctatttctcctccatgggagaaaatagataagagtagaaactgcagggaaaattatttgcataacaattcctctactaacaatcagctccttcctggagactgcccagctaaagcaatatgcatttaaatacagtcttccatttgcaagggaaaagtctcttgtaatccgaatctctttttgctttcgaactgctagtcaagtgcgtccacgagctgtttactagggatccctcatctgtccctccgggacctggtgctgcctctacctgacactcccttgggctccctgtaacctcttcagaggccctcgctgccagctctgtatcaggacccagaggaaggggccagaggctcgttgactggctgtgtgttgggattgagtctgtgccacgtgtttgtgctgtggtgtgtccccctctgtccaggcactgagataccagcgaggaggctccagagggcactctgcttgttattagagattacctcctgagaaaaaaggttccgcttggagcagaggggctgaatagcagaaggttgcacctcccccaaccttagatgttctaagtctttccattggatctcattggacccttccatggtgtgatcgtctgactggtgttatcaccgtgggctccctgactgggagttgatcgcctttcccaggtgctacacccttttccagctggatgagaatttgagtgctctgatccctctacagagcttccctgactcattctgaaggagccccattcctgggaaatattccctagaaacttccaaatcccctaagcagaccactgataaaaccatgtagaaaatttgttattttgcaacctcgctggactctcagtctctgagcagtgaatgattcagtgttaaatgtgatgaatactgtattttgtattgtttcaattgcatctcccagataatgtgaaaatggtccaggagaaggccaattcctatacgcagcgtgctttaaaaaataaataagaaacaactctttgagaaacaacaatttctactttgaagtcataccaatgaaaaaatgtatatgcacttataattttcctaataaagttctgtactcaaatgtagccaccaa

SEQ ID NO: 3 - DNA, 471 bp, Homo sapiens
gcactttcactctccgtcagccgcattgcccgctcggcgtccggcccccgacccgcgctcgtccgcccgcccgcccgcccgcccgcgccatgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaaggggcgcagagaagaaaaagtggggaaaaaagaaaagataggaaaaaagaagcgacagaagaagagaaaggctgcccagaaaaggaaaaactagttatctgccacctcgagatgga

SEQ ID NO: 4 - DNA, 1912 bp, Homo sapiens
ttttttttcttccctctagtgggcggggcagaggagttagccaagatgtgactttgaaaccctcagcgtctcagtgcccttttgttctaaacaaagaattttgtaattggttctaccaaagaaggatataatgaagtcactatgggaaaagatggggaggagagttgtaggattctacattaattctcttgtgcccttagcccactacttcagaatttcctgaagaaagcaagcctgaattggttttttaaattgctttaaaaattttttttaactgggttaatgcttgctgaattggaagtgaatgtccattcctttgcctcttttgcagatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaacacagatgtaaaagacttttttttatacgataaataacttttttttaagttacacatttttcagatataaaagactgaccaatattgtacagtttttattgcttgttggatttttgtcttgtgtttctttagtttttgtgaagtttaattgacttatttatataaattttttttgtttcatattgatgtgtgtctaggcaggacctgtggccaagttcttagttgctgtatgtctcgtggtaggactgtagaaaagggaactgaacattccagagcgtgtagtgaatcacgtaaagctagaaatgatccccagctgtttatgcatagataatctctccattcccgtggaacgtttttcctgttcttaagacgtgattttgctgtagaagatggcacttataaccaaagcccaaagtggtatagaaatgctggtttttcagttttcaggagtgggttgatttcagcacctacagtgtacagtcttgtattaagttgttaataaaagtacatgttaaacttaaaaaaaaaaaaaaaaaa

SEQ ID NO: 5 - DNA, 1691 bp, Homo sapiens
aacttcagtttgttggctgcggcagcaggtagcaaagtgacgccgagggcctgagtgctccagtagccaccgcatctggagaaccagcggttaccatggaggggatcagtatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaacacagatgtaaaagacttttttttatacgataaataacttttttttaagttacacatttttcagatataaaagactgaccaatattgtacagtttttattgcttgttggatttttgtcttgtgtttctttagtttttgtgaagtttaattgacttatttatataaattttttttgtttcatattgatgtgtgtctaggcaggacctgtggccaagttcttagttgctgtatgtctcgtggtaggactgtagaaaagggaactgaacattccagagcgtgtagtgaatcacgtaaagctagaaatgatccccagctgtttatgcatagataatctctccattcccgtggaacgtttttcctgttcttaagacgtgattttgctgtagaagatggcacttataaccaaagcccaaagtggtatagaaatgctggtttttcagttttcaggagtgggttgatttcagcacctacagtgtacagtcttgtattaagttgttaataaaagtacatgttaaacttaaaaaaaaaaaaaaaaaa

SEQ ID NO: 6 - DNA, 19 bp, Artificial
  Target Sequence
ataactacaccgaggaaat

SEQ ID NO: 7 - DNA, 21 bp, Artificial
  Sense strand with 3′NN
auaacuacaccgaggaaaunn

SEQ ID NO: 8 - DNA, 21 bp, Artificial
  Antisense strand with 3′NN
auuuccucgguguaguuaunn

SEQ ID NO: 9 - RNA, 21 nt, Artificial
  Sense Strand
auaacuacaccgaggaaauuu

SEQ ID NO: 10 - RNA, 21 nt, Artificial
  Antisense Strand
auuuccucgguguaguuauuu

SEQ ID NO: 11 - RNA, 19 nt, Artificial
  Sense Strand
auaacuacaccgaggaaau

SEQ ID NO: 12 - RNA, 19 nt, Artificial
  Antisense Strand
auuuccucgguguaguuau

SEQ ID NO: 13 - DNA, 48 bp, Artificial
  Hairpin duplex with loop
auaacuacaccgaggaaaunnnnnnnnauuuccucgguguaguuauuu

SEQ ID NO: 14 - DNA, 25 bp, Artificial
  Sense Strand
ataactacaccgaggaaatgggctc

SEQ ID NO: 15 - RNA, 25 nt, Artificial
  Sense Strand
auaacuacaccgaggaaaugggcuc

SEQ ID NO: 16 - RNA, 27 nt, Artificial
  Antisense Strand
gagcccauuuccucgguguaguuauuu

SEQ ID NO: 17 - DNA, 19 bp, Artificial
  Target Sequence
cgaaagccatgttgccaga

SEQ ID NO: 18 - DNA, 19 bp, Artificial
  Target Sequence
agacaagtgtgcattgacc

SEQ ID NO: 19 - DNA, 19 bp, Artificial
  Target Sequence
aaagtggattcaggagtac

SEQ ID NO: 20 - DNA, 19 bp, Artificial
  Target Sequence
ggattcaggagtacctgga

SEQ ID NO: 21 - DNA, 19 bp, Artificial
  Target Sequence
gattcttcgaaagccatgt

SEQ ID NO: 22 - DNA, 19 bp, Artificial
  Target Sequence
cagagccaacgtcaagcat

SEQ ID NO: 23 - DNA, 19 bp, Artificial
  Target Sequence
gagtacctggagaaagctt

SEQ ID NO: 24 - DNA, 19 bp, Artificial
  Target Sequence
tgaggtttgccagcattta

SEQ ID NO: 25 - DNA, 19 bp, Artificial
  Target Sequence
ggtatgatattgcagctta

SEQ ID NO: 26 - DNA, 19 bp, Artificial
  Target Sequence
gttagttccttcatgatca

SEQ ID NO: 27 - DNA, 19 bp, Artificial
  Target Sequence
ccttcatgatcatcacaat

SEQ ID NO: 28 - DNA, 19 bp, Artificial
  Target Sequence
acagtcaggtggtggctta

SEQ ID NO: 29 - DNA, 19 bp, Artificial
  Target Sequence
gtcaggtggtggcttaaca

SEQ ID NO: 30 - DNA, 19 bp, Artificial
  Target Sequence
ccatgtagaagccactatt

SEQ ID NO: 31 - DNA, 19 bp, Artificial
  Target Sequence
catgtagaagccactatta

SEQ ID NO: 32 - DNA, 19 bp, Artificial
  Target Sequence
agacaagtctttacagaat

SEQ ID NO: 33 - DNA, 19 bp, Artificial
  Target Sequence
atctgagagctcgctttga

SEQ ID NO: 34 - DNA, 19 bp, Artificial
  Target Sequence
gccatggaggcactaacaa

SEQ ID NO: 35 - DNA, 19 bp, Artificial
  Target Sequence
ccatggaggcactaacaaa

SEQ ID NO: 36 - DNA, 19 bp, Artificial
  Target Sequence
tccgaaatcagaagcgaaa

SEQ ID NO: 37 - DNA, 19 bp, Artificial
  Target Sequence
ggtgacatttccatgcata

SEQ ID NO: 38 - DNA, 19 bp, Artificial
  Target Sequence
gtgacatttccatgcataa

SEQ ID NO: 39 - DNA, 19 bp, Artificial
  Target Sequence
gacatttccatgcataaat

SEQ ID NO: 40 - DNA, 19 bp, Artificial
  Target Sequence
tatttgaagtggagccata

SEQ ID NO: 41 - DNA, 19 bp, Artificial
  Target Sequence
atctcaaactactggcaat

SEQ ID NO: 42 - DNA, 19 bp, Artificial
  Target Sequence
actactggcaatttgtaaa

SEQ ID NO: 43 - DNA, 19 bp, Artificial
  Target Sequence
gctaattgtttcatggtat

SEQ ID NO: 44 - DNA, 19 bp, Artificial
  Target Sequence
gtataaacgtcctactgta

SEQ ID NO: 45 - DNA, 19 bp, Artificial
  Target Sequence
tataaacgtcctactgtat

SEQ ID NO: 46 - DNA, 19 bp, Artificial
  Target Sequence
taaacgtcctactgtatgt

SEQ ID NO: 47 - DNA, 19 bp, Artificial
  Target Sequence
tgttagtgatggagcttaa

SEQ ID NO: 48 - DNA, 19 bp, Artificial
  Target Sequence
gttagtgatggagcttaaa

SEQ ID NO: 49 - DNA, 19 bp, Artificial
  Target Sequence
ttgcacactttgccatatt

SEQ ID NO: 50 - DNA, 19 bp, Artificial
  Target Sequence
tgcacactttgccatattt

SEQ ID NO: 51 - DNA, 19 bp, Artificial
  Target Sequence
cttagactaaggccattat

SEQ ID NO: 52 - DNA, 19 bp, Artificial
  Target Sequence
tccacgttctgctcatcat

SEQ ID NO: 53 - DNA, 19 bp, Artificial
  Target Sequence
ccacgttctgctcatcatt

SEQ ID NO: 54 - DNA, 19 bp, Artificial
  Target Sequence
acgttctgctcatcattct

SEQ ID NO: 55 - DNA, 19 bp, Artificial
  Target Sequence
aaacaagcattcacaactt

SEQ ID NO: 56 - DNA, 19 bp, Artificial
  Target Sequence
acaggacatttctctaaga

SEQ ID NO: 57 - DNA, 19 bp, Artificial
  Target Sequence
agtcgatagagctgtatta

SEQ ID NO: 58 - DNA, 19 bp, Artificial
  Target Sequence
gtcgatagagctgtattaa

SEQ ID NO: 59 - DNA, 19 bp, Artificial
  Target Sequence
agctgtattaagccactta

SEQ ID NO: 60 - DNA, 19 bp, Artificial
  Target Sequence
tcattcagtcttacgaata

SEQ ID NO: 61 - DNA, 19 bp, Artificial
  Target Sequence
tgattaaagactccagtta

SEQ ID NO: 62 - DNA, 19 bp, Artificial
  Target Sequence
gattaaagactccagttaa

SEQ ID NO: 63 - DNA, 19 bp, Artificial
  Target Sequence
atttcaaattggagcttca

SEQ ID NO: 64 - DNA, 19 bp, Artificial
  Target Sequence
tcaatccagctatgttata

SEQ ID NO: 65 - DNA, 19 bp, Artificial
  Target Sequence
ctctcactataccagtata

SEQ ID NO: 66 - DNA, 19 bp, Artificial
  Target Sequence
ggcagtcattatccaggta

SEQ ID NO: 67 - DNA, 19 bp, Artificial
  Target Sequence
cctaagcagaccactgata

SEQ ID NO: 68 - DNA, 19 bp, Artificial
  Target Sequence
gagaaggccaattcctata

SEQ ID NO: 69 - DNA, 19 bp, Artificial
  Target Sequence
ctatacgcagcgtgcttta

SEQ ID NO: 70 - DNA, 19 bp, Artificial
  Target Sequence
tatacgcagcgtgctttaa

SEQ ID NO: 71 - DNA, 19 bp, Artificial
  Target Sequence
gaaacaactctttgagaaa

SEQ ID NO: 72 - DNA, 19 bp, Artificial
  Target Sequence
tagttatctgccacctcga

SEQ ID NO: 73 - DNA, 19 bp, Artificial
  Target Sequence
gttatctgccacctcgaga

SEQ ID NO: 74 - DNA, 19 bp, Artificial
  Target Sequence
ttatctgccacctcgagat

SEQ ID NO: 75 - DNA, 19 bp, Artificial
  Target Sequence
tcttcttaactggcattgt

SEQ ID NO: 76 - DNA, 19 bp, Artificial
  Target Sequence
gattggtcatcctggtcat

SEQ ID NO: 77 - DNA, 19 bp, Artificial
  Target Sequence
ctgagaagcatgacggaca

SEQ ID NO: 78 - DNA, 19 bp, Artificial
  Target Sequence
tgagaagcatgacggacaa

SEQ ID NO: 79 - DNA, 19 bp, Artificial
  Target Sequence
gaagcatgacggacaagta

SEQ ID NO: 80 - DNA, 19 bp, Artificial
  Target Sequence
cagttgatgccgtggcaaa

SEQ ID NO: 81 - DNA, 19 bp, Artificial
  Target Sequence
tatgcaaggcagtccatgt

SEQ ID NO: 82 - DNA, 19 bp, Artificial
  Target Sequence
gcagtccatgtcatctaca

SEQ ID NO: 83 - DNA, 19 bp, Artificial
  Target Sequence
catctttgccaacgtcagt

SEQ ID NO: 84 - DNA, 19 bp, Artificial
  Target Sequence
atatctgtgaccgcttcta

SEQ ID NO: 85 - DNA, 19 bp, Artificial
  Target Sequence
catcatggttggccttatc

SEQ ID NO: 86 - DNA, 19 bp, Artificial
  Target Sequence
ctgtcctgctattgcatta

SEQ ID NO: 87 - DNA, 19 bp, Artificial
  Target Sequence
tgtcctgctattgcattat

SEQ ID NO: 88 - DNA, 19 bp, Artificial
  Target Sequence
tatcatctccaagctgtca

SEQ ID NO: 89 - DNA, 19 bp, Artificial
  Target Sequence
gttggctgccttactacat

SEQ ID NO: 90 - DNA, 19 bp, Artificial
  Target Sequence
ttcttccactgttgtctga

SEQ ID NO: 91 - DNA, 19 bp, Artificial
  Target Sequence
tcttccactgttgtctgaa

SEQ ID NO: 92 - DNA, 19 bp, Artificial
  Target Sequence
atctgtttccactgagtct

SEQ ID NO: 93 - DNA, 19 bp, Artificial
  Target Sequence
gactgaccaatattgtaca

SEQ ID NO: 94 - DNA, 19 bp, Artificial
  Target Sequence
attgatgtgtgtctaggca

SEQ ID NO: 95 - DNA, 19 bp, Artificial
  Target Sequence
gtctcgtggtaggactgta

SEQ ID NO: 96 - DNA, 19 bp, Artificial
  Target Sequence
ctcgtggtaggactgtaga

SEQ ID NO: 97 - DNA, 19 bp, Artificial
  Target Sequence
tcgtggtaggactgtagaa

SEQ ID NO: 98 - DNA, 19 bp, Artificial
  Target Sequence
gaacattccagagcgtgta

SEQ ID NO: 99 - DNA, 19 bp, Artificial
  Target Sequence
gtgtagtgaatcacgtaaa

SEQ ID NO: 100 - DNA, 19 bp, Artificial
  Target Sequence
tgaatcacgtaaagctaga

SEQ ID NO: 101 - DNA, 19 bp, Artificial
  Target Sequence
gaatcacgtaaagctagaa

SEQ ID NO: 102 - DNA, 19 bp, Artificial
  Target Sequence
atcacgtaaagctagaaat

SEQ ID NO: 103 - DNA, 19 bp, Artificial
  Target Sequence
tcacgtaaagctagaaatg

SEQ ID NO: 104 - DNA, 19 bp, Artificial
  Target Sequence
cacgtaaagctagaaatga

SEQ ID NO: 105 - DNA, 19 bp, Artificial
  Target Sequence
acgtaaagctagaaatgat

SEQ ID NO: 106 - DNA, 19 bp, Artificial
  Target Sequence
tcctgttcttaagacgtga

SEQ ID NO: 107 - DNA, 19 bp, Artificial
  Target Sequence
cctgttcttaagacgtgat

SEQ ID NO: 108 - DNA, 19 bp, Artificial
  Target Sequence
ctgtagaagatggcactta

SEQ ID NO: 109 - DNA, 19 bp, Artificial
  Target Sequence
tgtagaagatggcacttat

SEQ ID NO: 110 - DNA, 19 bp, Artificial
  Target Sequence
gtagaagatggcacttata

SEQ ID NO: 111 - DNA, 19 bp, Artificial
  Target Sequence
tagaagatggcacttataa

SEQ ID NO: 112 - DNA, 19 bp, Artificial
  Target Sequence
gatggcacttataaccaaa

SEQ ID NO: 113 - DNA, 19 bp, Artificial
  Target Sequence
atggattggtcatcctggt

SEQ ID NO: 114 - DNA, 19 bp, Artificial
  Target Sequence
gctattgcattatcatctc

SEQ ID NO: 115 - DNA, 19 bp, Artificial
  Target Sequence
ccaagctgtcacactccaa

SEQ ID NO: 116 - DNA, 19 bp, Artificial
  Target Sequence
agcacgcactcacctctgt

SEQ ID NO: 117 - DNA, 19 bp, Artificial
  Target Sequence
tgctgtatgtctcgtggta

SEQ ID NO: 118 - DNA, 19 bp, Artificial
  Target Sequence
tgtagtgaatcacgtaaag

SEQ ID NO: 119 - DNA, 19 bp, Artificial
  Target Sequence
ggagagttgtaggattcta

SEQ ID NO: 120 - DNA, 19 bp, Artificial
  Target Sequence
gttgtaggattctacatta

SEQ ID NO: 121 - DNA, 19 bp, Artificial
  Target Sequence
tgctgaattggaagtgaat

SEQ ID NO: 122 - DNA, 19 bp, Artificial
  Target Sequence
tcagtatatacacttcaga

SEQ ID NO: 123 - DNA, 19 bp, Artificial
  Target Sequence
acgccaaggtcgtggtcgt

SEQ ID NO: 124 - DNA, 19 bp, Artificial
  Target Sequence
ccaaggtcgtggtcgtgct

SEQ ID NO: 125 - DNA, 19 bp, Artificial
  Target Sequence
ctacagatgcccatgccga

SEQ ID NO: 126 - DNA, 19 bp, Artificial
  Target Sequence
acagatgcccatgccgatt

SEQ ID NO: 127 - DNA, 19 bp, Artificial
  Target Sequence
ccatgccgattcttcgaaa

SEQ ID NO: 128 - DNA, 19 bp, Artificial
  Target Sequence
gccgattcttcgaaagcca

SEQ ID NO: 129 - DNA, 19 bp, Artificial
  Target Sequence
agccatgttgccagagcca

SEQ ID NO: 130 - DNA, 19 bp, Artificial
  Target Sequence
ttgccagagccaacgtcaa

SEQ ID NO: 131 - DNA, 19 bp, Artificial
  Target Sequence
ccagagccaacgtcaagca

SEQ ID NO: 132 - DNA, 19 bp, Artificial
  Target Sequence
gagccaacgtcaagcatct

SEQ ID NO: 133 - DNA, 19 bp, Artificial
  Target Sequence
ccaacgtcaagcatctcaa

SEQ ID NO: 134 - DNA, 19 bp, Artificial
  Target Sequence
caacgtcaagcatctcaaa

SEQ ID NO: 135 - DNA, 19 bp, Artificial
  Target Sequence
acgtcaagcatctcaaaat

SEQ ID NO: 136 - DNA, 19 bp, Artificial
  Target Sequence
cgtcaagcatctcaaaatt

SEQ ID NO: 137 - DNA, 19 bp, Artificial
  Target Sequence
tctcaaaattctcaacact

SEQ ID NO: 138 - DNA, 19 bp, Artificial
  Target Sequence
tctcaacactccaaactgt

SEQ ID NO: 139 - DNA, 19 bp, Artificial
  Target Sequence
caaactgtgcccttcagat

SEQ ID NO: 140 - DNA, 19 bp, Artificial
  Target Sequence
aaactgtgcccttcagatt

SEQ ID NO: 141 - DNA, 19 bp, Artificial
  Target Sequence
cggctgaagaacaacaaca

SEQ ID NO: 142 - DNA, 19 bp, Artificial
  Target Sequence
gctgaagaacaacaacaga

SEQ ID NO: 143 - DNA, 19 bp, Artificial
  Target Sequence
ctgaagaacaacaacagac

SEQ ID NO: 144 - DNA, 19 bp, Artificial
  Target Sequence
tgaagaacaacaacagaca

SEQ ID NO: 145 - DNA, 19 bp, Artificial
  Target Sequence
gaagaacaacaacagacaa

SEQ ID NO: 146 - DNA, 19 bp, Artificial
  Target Sequence
aagaacaacaacagacaag

SEQ ID NO: 147 - DNA, 19 bp, Artificial
  Target Sequence
gaacaacaacagacaagtg

SEQ ID NO: 148 - DNA, 19 bp, Artificial
  Target Sequence
caacaacagacaagtgtgc

SEQ ID NO: 149 - DNA, 19 bp, Artificial
  Target Sequence
acaacagacaagtgtgcat

SEQ ID NO: 150 - DNA, 19 bp, Artificial
  Target Sequence
caacagacaagtgtgcatt

SEQ ID NO: 151 - DNA, 19 bp, Artificial
  Target Sequence
acagacaagtgtgcattga

SEQ ID NO: 152 - DNA, 19 bp, Artificial
  Target Sequence
aagtgtgcattgacccgaa

SEQ ID NO: 153 - DNA, 19 bp, Artificial
  Target Sequence
gtgcattgacccgaagcta

SEQ ID NO: 154 - DNA, 19 bp, Artificial
  Target Sequence
tgcattgacccgaagctaa

SEQ ID NO: 155 - DNA, 19 bp, Artificial
  Target Sequence
gcattgacccgaagctaaa

SEQ ID NO: 156 - DNA, 19 bp, Artificial
  Target Sequence
gacccgaagctaaagtgga

SEQ ID NO: 157 - DNA, 19 bp, Artificial
  Target Sequence
acccgaagctaaagtggat

SEQ ID NO: 158 - DNA, 19 bp, Artificial
  Target Sequence
ccgaagctaaagtggattc

SEQ ID NO: 159 - DNA, 19 bp, Artificial
  Target Sequence
aagctaaagtggattcagg

SEQ ID NO: 160 - DNA, 19 bp, Artificial
  Target Sequence
agctaaagtggattcagga

SEQ ID NO: 161 - DNA, 19 bp, Artificial
  Target Sequence
gctaaagtggattcaggag

SEQ ID NO: 162 - DNA, 19 bp, Artificial
  Target Sequence
ctaaagtggattcaggagt

SEQ ID NO: 163 - DNA, 19 bp, Artificial
  Target Sequence
taaagtggattcaggagta

SEQ ID NO: 164 - DNA, 19 bp, Artificial
  Target Sequence
aggagtacctggagaaagc

SEQ ID NO: 165 - DNA, 19 bp, Artificial
  Target Sequence
agtacctggagaaagcttt

SEQ ID NO: 166 - DNA, 19 bp, Artificial
  Target Sequence
acctggagaaagctttaaa

SEQ ID NO: 167 - DNA, 19 bp, Artificial
  Target Sequence
ctggagaaagctttaaaca

SEQ ID NO: 168 - DNA, 19 bp, Artificial
  Target Sequence
tggagaaagctttaaacaa

SEQ ID NO: 169 - DNA, 19 bp, Artificial
  Target Sequence
ggagaaagctttaaacaag

SEQ ID NO: 170 - DNA, 19 bp, Artificial
  Target Sequence
agataactacaccgaggaa

SEQ ID NO: 171 - DNA, 19 bp, Artificial
  Target Sequence
gataactacaccgaggaaa

SEQ ID NO: 172 - DNA, 19 bp, Artificial
  Target Sequence
ggaaatgggctcaggggac

SEQ ID NO: 173 - DNA, 19 bp, Artificial
  Target Sequence
gggactatgactccatgaa

SEQ ID NO: 174 - DNA, 19 bp, Artificial
  Target Sequence
ccatgaaggaaccctgttt

SEQ ID NO: 175 - DNA, 19 bp, Artificial
  Target Sequence
ccgtgaagaaaatgctaat

SEQ ID NO: 176 - DNA, 19 bp, Artificial
  Target Sequence
taactggcattgtgggcaa

SEQ ID NO: 177 - DNA, 19 bp, Artificial
  Target Sequence
tcatcctggtcatgggtta

SEQ ID NO: 178 - DNA, 19 bp, Artificial
  Target Sequence
ggtcatgggttaccagaag

SEQ ID NO: 179 - DNA, 19 bp, Artificial
  Target Sequence
tcatgggttaccagaagaa

SEQ ID NO: 180 - DNA, 19 bp, Artificial
  Target Sequence
cagaagaaactgagaagca

SEQ ID NO: 181 - DNA, 19 bp, Artificial
  Target Sequence
agaagaaactgagaagcat

SEQ ID NO: 182 - DNA, 19 bp, Artificial
  Target Sequence
gaagaaactgagaagcatg

SEQ ID NO: 183 - DNA, 19 bp, Artificial
  Target Sequence
aagaaactgagaagcatga

SEQ ID NO: 184 - DNA, 19 bp, Artificial
  Target Sequence
gcaaggcagtccatgtcat

SEQ ID NO: 185 - DNA, 19 bp, Artificial
  Target Sequence
aggcagtccatgtcatcta

SEQ ID NO: 186 - DNA, 19 bp, Artificial
  Target Sequence
tcatcagtctggaccgcta

SEQ ID NO: 187 - DNA, 19 bp, Artificial
  Target Sequence
accaacagtcagaggccaa

SEQ ID NO: 188 - DNA, 19 bp, Artificial
  Target Sequence
caaggaagctgttggctga

SEQ ID NO: 189 - DNA, 19 bp, Artificial
  Target Sequence
aggaagctgttggctgaaa

SEQ ID NO: 190 - DNA, 19 bp, Artificial
  Target Sequence
tggctgaaaaggtggtcta

SEQ ID NO: 191 - DNA, 19 bp, Artificial
  Target Sequence
ctgaaaaggtggtctatgt

SEQ ID NO: 192 - DNA, 19 bp, Artificial
  Target Sequence
ccgacttcatctttgccaa

SEQ ID NO: 193 - DNA, 19 bp, Artificial
  Target Sequence
ccaacgtcagtgaggcaga

SEQ ID NO: 194 - DNA, 19 bp, Artificial
  Target Sequence
cagtgaggcagatgacaga

SEQ ID NO: 195 - DNA, 19 bp, Artificial
  Target Sequence
gtgaggcagatgacagata

SEQ ID NO: 196 - DNA, 19 bp, Artificial
  Target Sequence
gaggcagatgacagatata

SEQ ID NO: 197 - DNA, 19 bp, Artificial
  Target Sequence
gcacatcatggttggcctt

SEQ ID NO: 198 - DNA, 19 bp, Artificial
  Target Sequence
tgtcacactccaagggcca

SEQ ID NO: 199 - DNA, 19 bp, Artificial
  Target Sequence
actccaagggccaccagaa

SEQ ID NO: 200 - DNA, 19 bp, Artificial
  Target Sequence
cctcaagaccacagtcatc

SEQ ID NO: 201 - DNA, 19 bp, Artificial
  Target Sequence
ccttcatcctcctggaaat

SEQ ID NO: 202 - DNA, 19 bp, Artificial
  Target Sequence
cctggaaatcatcaagcaa

SEQ ID NO: 203 - DNA, 19 bp, Artificial
  Target Sequence
caagcaagggtgtgagttt

SEQ ID NO: 204 - DNA, 19 bp, Artificial
  Target Sequence
gggtgtgagtttgagaaca

SEQ ID NO: 205 - DNA, 19 bp, Artificial
  Target Sequence
ttgagaacactgtgcacaa

SEQ ID NO: 206 - DNA, 19 bp, Artificial
  Target Sequence
ggtccagcctcaagatcct

SEQ ID NO: 207 - DNA, 19 bp, Artificial
  Target Sequence
cctcaagatcctctccaaa

SEQ ID NO: 208 - DNA, 19 bp, Artificial
  Target Sequence
caaaggaaagcgaggtgga

SEQ ID NO: 209 - DNA, 19 bp, Artificial
  Target Sequence
aggaaagcgaggtggacat

SEQ ID NO: 210 - DNA, 19 bp, Artificial
  Target Sequence
aaagcgaggtggacattca

SEQ ID NO: 211 - DNA, 19 bp, Artificial
  Target Sequence
ccactgagtctgagtcttc

SEQ ID NO: 212 - DNA, 19 bp, Artificial
  Target Sequence
gagtctgagtcttcaagtt

SEQ ID NO: 213 - DNA, 19 bp, Artificial
  Target Sequence
agtctgagtcttcaagttt

(19) United States
(12) Patent Application Publication
(10) Pub. No.: US 2009/0105182 A1
(43) Pub. Date: Apr. 23, 2009

(54) RNAi-MEDIATED INHIBITION OF STROMAL CELL-DERIVED FACTOR 1-RELATED TARGETS FOR TREATMENT OF NEOVASCULARIZATION-RELATED CONDITIONS

(75) Inventors: Jon E. Chatterton, Fort Worth, TX (US); David P. Bingaman, Weatherford, TX (US)

Correspondence Address: ALCON, IP LEGAL, TB4-8, 6201 SOUTH FREEWAY, FORT WORTH, TX, 76134, US

(73) Assignee: ALCON RESEARCH, LTD., Fort Worth, TX (US)

(21) Appl. No.: 12/298,940

(22) PCT Filed: Jun. 1, 2007
(86) PCT No.: PCT/US2007/070269

§ 371 Date: Oct. 29, 2008

Related U.S. Application Data
(60) Provisional application No. 60/810,273, filed on Jun. 2, 2006.

(51) Int. Cl. A61K 31/7105

(52) U.S. Cl. 514/44

(57) ABSTRACT

RNA interference is provided for inhibition of stromal cell-derived factor 1 (SDF1)-related targets in pathologic neovascularization-related conditions, including those cellular changes resulting from the signal transduction activity of the SDF1 targets that lead directly or indirectly to ocular neovascularization, abnormal angiogenesis, retinal vascular permeability, retinal edema, diabetic retinopathy particularly proliferative diabetic retinopathy, diabetic macular edema, exudative age-related macular degeneration, sequela associated with retinal ischemia, and posterior segment neovascularization, for example.

### RELATED APPLICATION

The present application claims the benefit of co-pending U.S. Provisional Patent Application Ser. No. 60/810,273 filed Jun. 2, 2006, the text of which is specifically incorporated by reference herein.

### FIELD OF THE INVENTION

The present invention relates to the field of interfering RNA compositions for inhibition of expression of stromal cell-derived factor 1 (SDF1)-related targets in pathologic neovascularization (NV)-related conditions, including those cellular changes resulting from the signal transduction activity of the SDF1 targets that lead directly or indirectly to NV, ocular NV, abnormal angiogenesis, retinal vascular permeability, retinal edema, diabetic retinopathy particularly proliferative diabetic retinopathy, diabetic macular edema, exudative age-related macular degeneration, sequela associated with retinal ischemia, and posterior segment NV, for example.

### BACKGROUND OF THE INVENTION

Pathologic NV and related conditions occur as a cascade of events that progresses from an initiating stimulus to the formation of abnormal new capillaries. The stimulus appears to be the elaboration of various proangiogenic growth factors such as vascular endothelial growth factor (VEGF), platelet-derived growth factor (PDGF), and angiopoietins, among others. Following initiation of the angiogenic cascade, the capillary basement membrane and extracellular matrix are degraded and capillary endothelial cell proliferation and migration occur. Endothelial sprouts anastomose to form tubes with subsequent patent lumen formation. The new capillaries commonly have increased vascular permeability or leakiness due to immature barrier function, which can lead to tissue edema. Differentiation into a mature capillary is indicated by the presence of a continuous basement membrane and normal endothelial junctions between other endothelial cells and pericytes; however, this differentiation process is often impaired during pathologic conditions.

Retinal NV is observed in retinal ischemia, proliferative and nonproliferative diabetic retinopathy (PDR and NPDR, respectively), retinopathy of prematurity (ROP), central and branch retinal vein occlusion, and age-related macular degeneration (AMD). The retina includes choriocapillaries that form the choroid and are responsible for providing nourishment to the retina, Bruch's membrane that acts as a filter between the retinal pigment epithelium (RPE) and the choriocapillaries, and the RPE that secretes angiogenic and anti-angiogenic factors responsible for, among many other things, the growth and recession of blood vessels.

NV can include damage to Bruch's membrane which then allows growth factor to come in contact with the choriocapillaries and initiate the process of angiogenesis. The new capillaries can break through the RPE as well as Bruch's membrane to form a new vascular layer above the RPE. Leakage of the vascular layer leads to wet or exudative AMD and subsequent loss of cones and rods that are vital to vision.

Exudative AMD and PDR are the major causes of acquired blindness in developed countries and are characterized by pathologic posterior segment neovascularization (PSNV). The PSNV found in exudative AMD is characterized as pathologic choroidal NV, whereas PDR exhibits preretinal NV. In spite of the prevalence of PSNV, treatment strategies are few and palliative at best. Approved treatments for the PSNV in exudative AMD include laser photocoagulation and photodynamic therapy with VISUDYNE®; both therapies involve laser-induced occlusion of affected vasculature and are associated with localized laser-induced damage to the retina. For patients with PDR, grid or panretinal laser photocoagulation and surgical interventions, such as vitrectomy and removal of preretinal membranes, are the only options currently available. Several different compounds are being evaluated clinically for the pharmacologic treatment of PSNV, including RETAANE® (Alcon Research, Ltd.), Lucentis™, Avastin™ (Genentech), adPEDF (GenVec), squalamine (Genaera), CA4P (OxiGENE), VEGF trap (Regeneron), LY333531 (Lilly), and siRNAs targeting VEGF (Cand5, Acuity) and VEGFR-1 (Sirna-027, Sirna Therapeutics). Macugen® (Eyetech/Pfizer), an anti-VEGF aptamer injected intravitreally, has recently been approved for such use. In addition, an “Ang-trap” (Amgen) is in development to sequester the ligand for Tie-2 and an siRNA against RTP801, a downstream target of HIF-1, is under development (Quark Biotech).

Macular edema is the major cause of vision loss in diabetic patients, whereas preretinal NV (PDR) is the major cause of legal blindness. Diabetes mellitus is characterized by persistent hyperglycemia that produces reversible and irreversible pathologic changes within the microvasculature of various organs. Diabetic retinopathy (DR), therefore, is a retinal microvascular disease that is manifested as a cascade of stages with increasing levels of severity and worsening prognoses for vision. Major risk factors reported for developing diabetic retinopathy include the duration of diabetes mellitus, quality of glycemic control, and presence of systemic hypertension. DR is broadly classified into 2 major clinical stages: nonproliferative diabetic retinopathy (NPDR) and proliferative diabetic retinopathy (PDR), where the term “proliferative” refers to the presence of preretinal NV as previously stated.

Nonproliferative diabetic retinopathy (NPDR) and subsequent macular edema are associated, in part, with retinal ischemia that results from the retinal microvasculopathy induced by persistent hyperglycemia. NPDR encompasses a range of clinical subcategories which include initial “background” DR, where small multifocal changes are observed within the retina (e.g., microaneurysms, “dot-blot” hemorrhages, and nerve fiber layer infarcts), through preproliferative DR, which immediately precedes the development of PSNV. The histopathologic hallmarks of NPDR are retinal microaneurysms, capillary basement membrane thickening, endothelial cell and pericyte loss, and eventual capillary occlusion leading to regional ischemia. Data accumulated from animal models and empirical human studies show that retinal ischemia is often associated with increased local levels of proinflammatory and/or proangiogenic growth factors and cytokines, such as prostaglandin E2, vascular endothelial growth factor (VEGF), insulin-like growth factor-1 (IGF-1), Angiopoietin 2, etc. Diabetic macular edema can be seen during either NPDR or PDR, however, it often is observed in the latter stages of NPDR and is a prognostic indicator of progression towards development of the most severe stage, PDR.

At present, no pharmacologic therapy is approved for the treatment of NPDR and/or macular edema. The current standard of care is laser photocoagulation, which is used to stabilize or resolve macular edema and retard the progression toward PDR. Laser photocoagulation may reduce retinal ischemia by destroying healthy tissue and thereby decreasing metabolic demand; it also may modulate the expression and production of various cytokines and trophic factors. Similar to the exudative AMD treatments, laser photocoagulation in diabetic patients is a cytodestructive procedure and the visual field of the treated eye is irreversibly compromised. Other than diabetic macular edema, retinal edema can be observed in various other posterior segment diseases, such as posterior uveitis, branch retinal vein occlusion, surgically induced inflammation, endophthalmitis (sterile and non-sterile), scleritis, and episcleritis, etc.

Small molecule receptor tyrosine kinase (RTK) inhibitors (RTKi) such as PKC412 (CPG 41251), PTK787, and MAE 87 have been described that act on VEGF receptors and inhibit retinal NV or choroidal NV in mice. Each of these molecules inhibits multiple kinases. For example, PKC412 inhibits KDR (hVEGFR-2), PDGFR-β, Flk-1 (mVEGFR-2), and Flt-1 (VEGFR-1) as well as several PKC isotypes; PTK787 inhibits KDR and Flk-1 (human and murine VEGFR-2, respectively), VEGFR-1, PDGFR-β, c-Kit, and cFms; and MAE 87 inhibits VEGFR-2, IGF-1R, FGFR-1, and EGFR. Inhibition of multiple kinases may completely block NV, however, such inhibition is expected to have toxic side effects.

U.S. Patent Publication 2006/0019917, published Jan. 26, 2006, to Guerciolini et al. relates to RNA interference mediated inhibition of SDF-1 receptor gene expression. U.S. Patent Publication 2005/0124569, published Jun. 9, 2005, to Guerciolini et al. relates to RNA interference mediated inhibition of CXCR4 receptor gene expression. However, said publications teach none of the particular target sequences for RNA interference for uses as provided herein.

The present invention addresses the above-cited pathologies and provides alternate and improved compositions and methods using interfering RNAs that provide targeted specificity.

### SUMMARY OF THE INVENTION

The present invention overcomes these and other drawbacks of the prior art by providing highly potent and efficacious prevention or intervention of pathologic NV-related conditions. In one aspect, the methods of the invention include treating a NV-related condition by administering interfering RNAs that silence expression of SDF1 mRNA or mRNA encoding its receptor CXCR4 involved in a NV-related condition, thus decreasing SDF1/CXCR4 signaling and treating NV and related conditions by effecting a lowering of pre-angiogenic and angiogenic cellular activity.

A method of treating a NV-related condition in a subject in need thereof is an embodiment of the invention. The method comprises administering to the subject a composition comprising an effective amount of interfering RNA having a length of 19 to 49 nucleotides, and a pharmaceutically acceptable carrier, wherein the interfering RNA comprises a region of at least 13 contiguous nucleotides having at least 90% sequence complementarity to, or at least 90% sequence identity with, the penultimate 13 nucleotides of the 3′ end of an mRNA corresponding to any one of SEQ ID NO:6, SEQ ID NO:75-SEQ ID NO:122, and SEQ ID NO: 170-SEQ ID NO: 213. The NV-related condition is treated thereby.

In yet another embodiment of the invention, a method of attenuating expression of CXCR4 mRNA of the subject comprises administering to the subject a composition comprising an effective amount of interfering RNA having a length of 19 to 49 nucleotides and a pharmaceutically acceptable carrier and the interfering RNA comprises a sense nucleotide strand, an antisense nucleotide strand, and a region of at least near-perfect contiguous complementarity of at least 19 nucleotides where the antisense strand hybridizes under physiological conditions to a portion of mRNA corresponding to SEQ ID NO:4 beginning at nucleotide 122, 123, 137, 151, 163, 182, 244, 271, 278, 280, 291, 292, 293, 294, 421, 424, 481, 519, 535, 537, 547, 550, 604, 619, 626, 628, 630, 701, 772, 778, 806, 883, 893, 905, 912, 922, 1063, 1070, 1085, 1088, 1091, 1117, 1122, 1123, 345, 459, 486, 521, 522, 525, 600, 639, 647, 832, 867, 925, 962, 963, 979, 1068, 1190, 1191, 1330, 1448, 1557, 1607, 1609, 1610, 1638, 1652, 1658, 1659, 1661, 1662, 1663, 1664, 1732, 1733, 1756, 1757, 1758, 1759, 1764, 159, 164, 288, or to a portion of mRNA corresponding to SEQ ID NO:5 beginning at nucleotide 106. The expression of CXCR4 mRNA is attenuated thereby.

Another aspect of the invention is a method of attenuating expression of SDF1 mRNA of the subject, comprising administering to the subject a composition comprising an effective amount of interfering RNA having a length of 19 to 49 nucleotides and a pharmaceutically acceptable carrier, the interfering RNA comprising a sense nucleotide strand, an antisense nucleotide strand, and a region of at least near-perfect contiguous complementarity of at least 19 nucleotides; wherein the antisense strand hybridizes under physiological conditions to a portion of mRNA corresponding to SEQ ID NO:1 beginning at nucleotide 94, 97, 170, 172, 180, 184, 198, 205, 208, 211, 214, 215, 217, 218, 227, 236, 247, 248, 273, 275, 276, 277, 278, 279, 281, 284, 286, 287, 289, 295, 299, 300, 301, 306, 307, 309, 312, 313, 314, 315, 316, 328, 331, 334, 336, 337, 338, 194, 291, 317, 322, 187, 209, 330, 511, 551, 664, 671, 790, 793, 970, 971, 1267, 1301, 1358, 1359, 1388, 1492, 1493, 1495, 1653, 1701, 1708, 1842, 1857, 1858, 1860, 1913, or 1914, or to a portion of mRNA corresponding to SEQ ID NO:2 beginning at nucleotide 494, 495, 598, 996, 997, 999, 1506, 1695, 1783, 1784, 1792, 1862, 1892, 1893, 2081, 2155, 2249, 2284, 3225, 3386, 3400, 3401, or 3431, or to a portion of mRNA corresponding to SEQ ID NO:3 beginning at nucleotide 447, 449, or 450. The expression of SDF1 mRNA is attenuated thereby.

A method of treating a NV-related condition in a subject in need thereof is an embodiment of the invention, the method comprising administering to the subject a composition comprising an effective amount of interfering RNA having a length of 19 to 49 nucleotides, and a pharmaceutically acceptable carrier, the interfering RNA comprising a sense nucleotide strand, an antisense nucleotide strand, and a region of at least near-perfect contiguous complementarity of at least 19 nucleotides; wherein the antisense strand hybridizes under physiological conditions to a portion of mRNA corresponding to SEQ ID NO:4 comprising nucleotide 122, 123, 137, 151, 163, 182, 244, 271, 278, 280, 291, 292, 293, 294, 421, 424, 481, 519, 535, 537, 547, 550, 604, 619, 626, 628, 630, 701, 772, 778, 806, 883, 893, 905, 912, 922, 1063, 1070, 1085, 1088, 1091, 1117, 1122, 1123, 345, 459, 486, 521, 522, 525, 600, 639, 647, 832, 867, 925, 962, 963, 979, 1068, 1190, 1191, 1330, 1448, 1557, 1607, 1609, 1610, 1638, 1652, 1658, 1659, 1661, 1662, 1663, 1664, 1732, 1733, 1756, 1757, 1758, 1759, 1764, 483, 969, 987, 1257, 1599, 1653, 159, 164, or 288 or to a portion of mRNA corresponding to SEQ ID NO:5 comprising nucleotide 106. The NV-related condition is treated thereby.

A method of treating a NV-related condition in a subject in need thereof, comprising administering to the subject a composition comprising an effective amount of interfering RNA having a length of 19 to 49 nucleotides, and a pharmaceutically acceptable carrier, the interfering RNA comprising a sense nucleotide strand, an antisense nucleotide strand, and a region of at least near-perfect contiguous complementarity of at least 19 nucleotides, wherein the antisense strand hybridizes under physiological conditions to a portion of mRNA corresponding to SEQ ID NO:1 beginning at nucleotide 94, 97, 170, 172, 180, 184, 198, 205, 208, 211, 214, 215, 217, 218, 227, 236, 247, 248, 273, 275, 276, 277, 278, 279, 281, 284, 286, 287, 289, 295, 299, 300, 301, 306, 307, 309, 312, 313, 314, 315, 316, 328, 331, 334, 336, 337, 338, 194, 291, 317, 322, 187, 209, 330, 511, 551, 664, 671, 790, 793, 970, 971, 1267, 1301, 1358, 1359, 1388, 1492, 1493, 1495, 1653, 1701, 1708, 1842, 1857, 1858, 1860, 1913, or 1914, or to a portion of mRNA corresponding to SEQ ID NO:2 beginning at nucleotide 494, 495, 598, 996, 997, 999, 1506, 1695, 1783, 1784, 1792, 1862, 1892, 1893, 2081, 2155, 2249, 2284, 3225, 3386, 3400, 3401, or 3431, or to a portion of mRNA corresponding to SEQ ID NO:3 beginning at nucleotide 447, 449, or 450 is an aspect of the invention. The NV-related condition is treated thereby.

A second interfering RNA as defined by the sets of interfering RNAs as cited above may also be administered to the subject in a further embodiment of the invention. The second interfering RNA may target the SDF1 and/or the CXCR4 mRNA, i.e., both first and second interfering RNAs may target the same mRNA or different mRNAs. In further embodiments, a third, fourth, or more interfering RNAs as defined by the sets of interfering RNAs as cited above may be administered.

A further embodiment of the invention is a method of treating a NV-related condition in a subject in need thereof, where the method comprises administering to the subject a composition comprising a double stranded siRNA molecule that down regulates expression of a CXCR4 gene via RNA interference, wherein each strand of the siRNA molecule is independently about 19 to about 27 nucleotides in length; and one strand of the siRNA molecule comprises a nucleotide sequence having substantial complementarity to an mRNA corresponding to the CXCR4 gene, so that the siRNA molecule directs cleavage of the mRNA via RNA interference.

A method of attenuating expression of CXCR4 mRNA of a subject, comprising administering to the subject a composition comprising an effective amount of a single-stranded interfering RNA having a length of 19 to 49 nucleotides, and a pharmaceutically acceptable carrier is a further embodiment of the invention. For this embodiment, the single-stranded interfering RNA hybridizes under physiological conditions to a portion of mRNA corresponding to SEQ ID NO:4 comprising nucleotide 122, 123, 137, 151, 163, 182, 244, 271, 278, 280, 291, 292, 293, 294, 421, 424, 481, 519, 535, 537, 547, 550, 604, 619, 626, 628, 630, 701, 772, 778, 806, 883, 893, 905, 912, 922, 1063, 1070, 1085, 1088, 1091, 1117, 1122, 1123, 345, 459, 486, 521, 522, 525, 600, 639, 647, 832, 867, 925, 962, 963, 979, 1068, 1190, 1191, 1330, 1448, 1557, 1607, 1609, 1610, 1638, 1652, 1658, 1659, 1661, 1662, 1663, 1664, 1732, 1733, 1756, 1757, 1758, 1759, 1764, 159, 164, or 288, or a portion of mRNA corresponding to SEQ ID NO:5 beginning at nucleotide 106, and the interfering RNA has a region of at least near-perfect contiguous complementarity with the hybridizing portion of mRNA corresponding to SEQ ID NO:4 or SEQ ID NO:5, respectively. The expression of CXCR4 mRNA is thereby attenuated.

The invention includes as a further embodiment a composition comprising an interfering RNA consisting essentially of a nucleotide sequence corresponding to any one of SEQ ID NO:17-SEQ ID NO:74 and SEQ ID NO:123-SEQ ID NO: 169, or a complement thereof; and a pharmaceutically acceptable carrier.

A composition comprising an interfering RNA consisting essentially of a nucleotide sequence corresponding to any one of SEQ ID NO:6, SEQ ID NO:75-SEQ ID NO:112, SEQ ID NO: 170-SEQ ID NO: 213, and SEQ ID NO:119-SEQ ID NO:122, or a complement thereof; and a pharmaceutically acceptable carrier is another embodiment of the invention.

Use of any of the embodiments as described herein in the preparation of a medicament for attenuating expression of SDF1 mRNA or of CXCR4 mRNA and thereby treating a NV-related condition as set forth herein is also an embodiment of the present invention.

### DETAILED DESCRIPTION OF THE INVENTION

The references cited herein, to the extent that they provide exemplary procedural or other details supplementary to those set forth herein, are specifically incorporated by reference.

Those of skill in the art, in light of the present disclosure, will appreciate that obvious modifications of the embodiments disclosed herein can be made without departing from the spirit and scope of the invention. All of the embodiments disclosed herein can be made and executed without undue experimentation in light of the present disclosure. The full scope of the invention is set out in the disclosure and equivalent embodiments thereof. The specification should not be construed to unduly narrow the full scope of protection to which the present invention is entitled.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{rd} Edition.

As used herein and unless otherwise indicated, the terms “a” and “an” are taken to mean “one”, “at least one” or “one or more”.

The term “siRNA” as used herein refers to a double-stranded interfering RNA unless otherwise noted. Examples of RNA-like molecules that can interact with RISC include RNA molecules containing one or more chemically modified nucleotides, one or more deoxyribonucleotides, and/or one or more non-phosphodiester linkages. For purposes of the present discussion, all RNA or RNA-like molecules that can interact with RISC and participate in RISC-mediated changes in gene expression will be referred to as “interfering RNAs.” SiRNAs, shRNAs, miRNAs, and dicer-substrate 27-mer duplexes are, therefore, subsets of “interfering RNAs.”

The term “a neovascularization-related condition,” as used herein, includes pre-angiogenic conditions and angiogenic conditions, and includes those cellular changes resulting from the expression of SDF1 mRNA or CXCR4 mRNA that leads directly or indirectly to NV and related conditions. The interfering RNAs of the invention are useful for treating patients with NV, ocular NV, abnormal angiogenesis, retinal vascular permeability, retinal edema, diabetic retinopathy particularly proliferative diabetic retinopathy, diabetic macular edema, exudative age-related macular degeneration, sequela associated with retinal ischemia, and posterior segment NV, or patients at risk of developing such conditions, for example. The SDF1- or CXCR4-specific interfering RNAs provide for treatment of NV while avoiding toxic side effects due to less specific treatments.

“Near-perfect,” as used herein, means the antisense strand of the siRNA is “substantially complementary to,” and the sense strand of the siRNA is “substantially identical” to at least a portion of the target mRNA. “Identity,” as known by one of ordinary skill in the art, is the degree of sequence relatedness between nucleotide sequences as determined by matching the order and identity of nucleotides between the sequences. In one embodiment, the antisense strand of an siRNA having 80% and between 80% up to 100% complementarity, for example, 85%, 90% or 95% complementarity, to the target mRNA sequence are considered near-perfect complementarity and may be used in the present invention. “Perfect” contiguous complementarity is standard Watson-Crick base pairing of adjacent base pairs. “At least near-perfect” contiguous complementarity includes “perfect” complementarity as used herein. Computer methods for determining identity or complementarity are designed to identify the greatest degree of matching of nucleotide sequences, for example, BLASTN (Altschul, S. F., et al. (1990) J. Mol. Biol. 215:403-410).

The term “percent identity” describes the percentage of contiguous nucleotides in a first nucleic acid molecule that is the same as in a set of contiguous nucleotides of the same length in a second nucleic acid molecule. The term “percent complementarity” describes the percentage of contiguous nucleotides in a first nucleic acid molecule that can base pair in the Watson-Crick sense with a set of contiguous nucleotides in a second nucleic acid molecule.

“Hybridization” refers to a process in which single-stranded nucleic acids with complementary or near-complementary base sequences interact to form hydrogen-bonded complexes called hybrids. Hybridization reactions are sensitive and selective. In vitro, the specificity of hybridization (i.e., stringency) is controlled by the concentrations of salt or formamide in prehybridization and hybridization solutions, for example, and by the hybridization temperature; such procedures are well known in the art. In particular, stringency is increased by reducing the concentration of salt, increasing the concentration of formamide, or raising the hybridization temperature.

For example, high stringency conditions could occur at about 50% formamide at 37° C. to 42° C. Reduced stringency conditions could occur at about 35% to 25% formamide at 30° C. to 35° C. Examples of stringency conditions for hybridization are provided in Sambrook, J., 1989, Molecular Cloning: A Laboratory Manual, Cold Spring Harbor Laboratory Press, Cold Spring Harbor, N.Y. Further examples of stringent hybridization conditions include 400 mM NaCl, 40 mM PIPES pH 6.4, 1 mM EDTA, 50° C. or 70° C. for 12-16 hours followed by washing, or hybridization at 70° C. in 1×SSC or 50° C. in 1×SSC, 50% formamide followed by washing at 70° C. in 0.3×SSC, or hybridization at 70° C. in 4×SSC or 50° C. in 4×SSC, 50% formamide followed by washing at 67° C. in 1×SSC. The temperature for hybridization is about 5-10° C. less than the melting temperature (Tₘ) of the hybrid where Tₘ is determined for hybrids between 19 and 49 base pairs in length using the following calculation: Tₘ ° C.=81.5+16.6(log₁₀[Na+])+0.41(% G+C)−(600/N) where N is the number of bases in the hybrid, and [Na+] is the concentration of sodium ions in the hybridization buffer.

RNA interference (RNAi) is a process by which double-stranded RNA (dsRNA) is used to silence gene expression. While not wanting to be bound by theory, RNAi begins with the cleavage of longer dsRNAs into small interfering RNAs (siRNAs) by an RNaseIII-like enzyme, dicer. SiRNAs are dsRNAs that are usually about 19 to 28 nucleotides, or 20 to 25 nucleotides, or 21 to 22 nucleotides in length and often contain 2-nucleotide 3′ overhangs, and 5′ phosphate and 3′ hydroxyl termini. One strand of the siRNA is incorporated into a ribonucleoprotein complex known as the RNA-induced silencing complex (RISC). RISC uses this siRNA strand to identify mRNA molecules that are at least partially complementary to the incorporated siRNA strand, and then cleaves these target mRNAs or inhibits their translation. Therefore, the siRNA strand that is incorporated into RISC is known as the guide strand or the antisense strand. The other siRNA strand, known as the passenger strand or the sense strand, is eliminated from the siRNA and is at least partially homologous to the target mRNA. Those of skill in the art will recognize that, in principle, either strand of an siRNA can be incorporated into RISC and function as a guide strand. However, siRNA design (e.g., decreased siRNA duplex stability at the 5′ end of the desired guide strand) can favor incorporation of the desired guide strand into RISC.

RISC-mediated cleavage of mRNAs having a sequence at least partially complementary to the guide strand leads to a decrease in the steady state level of that mRNA and of the corresponding protein encoded by this mRNA. Alternatively, RISC can also decrease expression of the corresponding protein via translational repression without cleavage of the target mRNA. Other RNA molecules and RNA-like molecules can also interact with RISC and silence gene expression. Examples of other RNA molecules that can interact with RISC include short hairpin RNAs (shRNAs), single-stranded siRNAs, microRNAs (miRNAs), and dicer-substrate 27-mer duplexes.

Nucleic acid sequences cited herein are written in a 5′ to 3′ direction unless indicated otherwise. The term “nucleic acid,” as used herein, refers to either DNA or RNA or a modified form thereof comprising the purine or pyrimidine bases present in DNA (adenine “A,” cytosine “C,” guanine “G,” thymine “T”) or in RNA (adenine “A,” cytosine “C,” guanine “G,” uracil “U”). Interfering RNAs provided herein may comprise “T” bases, particularly at 3′ ends, even though “T” bases do not naturally occur in RNA. “Nucleic acid” includes the terms “oligonucleotide” and “polynucleotide” and can refer to a single-stranded molecule or a double-stranded molecule. A double-stranded molecule is formed by Watson-Crick base pairing between A and T bases, C and G bases, and between A and U bases. The strands of a double-stranded molecule may have partial, substantial or full complementarity to each other and will form a duplex hybrid, the strength of bonding of which is dependent upon the nature and degree of complementarity of the sequence of bases.

An mRNA sequence is readily deduced from the sequence of the corresponding DNA sequence. For example, SEQ ID NO:1 provides the sense strand sequence of DNA corresponding to the mRNA for SDF1, variant 1. The mRNA sequence is identical to the DNA sense strand sequence with the “T” bases replaced with “U” bases. Therefore, the mRNA sequence of SDF1, variant 1, is known from SEQ ID NO:1, for example.

Interfering RNA of embodiments of the invention appear to act in a catalytic manner for cleavage of target mRNA, i.e., interfering RNA is able to effect inhibition of target mRNA in substoichiometric amounts. As compared to antisense therapies, significantly less interfering RNA is required to provide a therapeutic effect under such cleavage conditions.

The present invention relates to the use of interfering RNA to inhibit the expression of stromal cell-derived factor 1 (SDF1, also known as CXCL12, designated herein as “SDF1”) mRNA or of chemokine (C—X—C motif) receptor 4 (CXCR4) mRNA. SDF1 recruits and activates lymphocytes and has important functions in the development and function of the immune system. SDF1 has also been shown to affect endothelial cells during angiogenesis. CXCR4, a seven-transmembrane G protein-coupled receptor best known as the co-receptor for HIV-1, is the only receptor for SDF1 and, thereby, mediates the chemotactic response of leukocytes to SDF1. SDF1/CXCR4 signaling has been shown to promote NV in cancer and in proliferative retinopathy.

According to the present invention, inhibiting the expression of SDF1 mRNA, CXCR4 mRNA, or both SDF1 and CXCR4 mRNAs effectively reduces the action of SDF1. Further, interfering RNAs as set forth herein provided exogenously or expressed endogenously are particularly effective at silencing SDF1 mRNA or CXCR4 mRNA.

Stromal Cell-Derived Factor 1 mRNA (SDF1 or CXCL12): The GenBank database provides the DNA sequence for SDF1 transcript variant 1 as accession no. NM_{—}199168, provided in the “Sequence Listing” as SEQ ID NO:1. SEQ ID NO:1 provides the sense strand sequence of DNA that corresponds to the mRNA encoding SDF1 variant 1 (with the exception of “T” bases for “U” bases). The coding sequence for SDF1 variant 1 is from nucleotides 90-359.

Equivalents of the above cited SDF1 variant 1 mRNA sequence are alternative splice forms, allelic forms, isozymes, or a cognate thereof. A cognate is a stromal cell-derived factor 1 variant 1 mRNA from another mammalian species that is homologous to SEQ ID NO:1 (i.e., an ortholog).

The GenBank database provides the DNA sequence for SDF1 transcript variant 2 as accession no. NM_{—}000609, provided in the “Sequence Listing” as SEQ ID NO:2. SEQ ID NO:2 provides the sense strand sequence of DNA that corresponds to the mRNA encoding SDF1 variant 2 (with the exception of “T” bases for “U” bases). The coding sequence for SDF1 variant 2 is from nucleotides 90-371.

Equivalents of the above cited SDF1 variant 2 mRNA sequence are alternative splice forms, allelic forms, isozymes, or a cognate thereof. A cognate is a stromal cell-derived factor 1 variant 2 mRNA from another mammalian species that is homologous to SEQ ID NO:2 (i.e., an ortholog).

The GenBank database provides the DNA sequence for SDF1 transcript variant 3 as accession no. NM_{—}001033886, provided in the “Sequence Listing” as SEQ ID NO:3. SEQ ID NO:3 provides the sense strand sequence of DNA that corresponds to the mRNA encoding SDF1 variant 3 (with the exception of “T” bases for “U” bases). The coding sequence for SDF1 variant 3 is from nucleotides 90-449.

Equivalents of the above cited SDF1 variant 3 miRNA sequence are alternative splice forms, allelic forms, isozymes, or a cognate thereof. A cognate is a stromal cell-derived factor 1 variant 3 mRNA from another mammalian species that is homologous to SEQ ID NO:3 (i.e., an ortholog).

Chemokine (C—X—C motif) receptor 4 Variant 1 and Variant 2 mRNA (CXCR4): CXCR4 variant 1 mRNA is a longer transcript and encodes a longer isoform as compared to variant 2. Variant 2 has a distinct 5′ UTR and lacks an in-frame portion of the 5′ coding region as compared to variant 1. Variant 2 therefore has a shorter N-terminus when compared to variant 1.

The GenBank database provides the DNA sequence for CXCR4 transcript variant 1 as accession no. NM_{—}001008540, provided in the “Sequence Listing” as SEQ ID NO:4. SEQ ID NO:4 provides the sense strand sequence of DNA that corresponds to the mRNA encoding CXCR4 variant 1 (with the exception of “T” bases for “U” bases). The coding sequence for CXCR4 variant 1 is from nucleotides 305-1375.

Equivalents of the above cited CXCR4 variant 1 mRNA sequence are alternative splice forms, allelic forms, isozymes, or a cognate thereof. A cognate is a stromal cell-derived factor 1 receptor 4 variant 1 mRNA from another mammalian species that is homologous to SEQ ID NO:4 (i.e., an ortholog).

The GenBank database provides the DNA sequence for CXCR4 transcript variant 2 as accession no. NM_{—}003467, provided in the “Sequence Listing” as SEQ ID NO:5. SEQ ID NO:5 provides the sense strand sequence of DNA that corresponds to the mRNA encoding CXCR4 variant 2 (with the exception of “T” bases for “U” bases). The coding sequence for CXCR4 variant 2 is from nucleotides 96-1154.

Equivalents of the above cited CXCR4 variant 2 mRNA sequence are alternative splice forms, allelic forms, isozymes, or a cognate thereof. A cognate is a stromal cell-derived factor 1 receptor 4 variant 2 mRNA from another mammalian species that is homologous to SEQ ID NO:5 (i.e., an ortholog).

Attenuating expression of an mRNA: The phrase, “attenuating expression of an mRNA,” as used herein, means administering or expressing an amount of interfering RNA (e.g., an siRNA) to reduce translation of the target mRNA into protein, either through mRNA cleavage or through direct inhibition of translation. The reduction in expression of the target mRNA or the corresponding protein is commonly referred to as “knock-down” and is reported relative to levels present following administration or expression of a non-targeting control RNA (e.g., a non-targeting control siRNA). Knock-down of expression of an amount including and between 50% and 100% is contemplated by embodiments herein. However, it is not necessary that such knock-down levels be achieved for purposes of the present invention. In one embodiment, a single interfering RNA targeting SDF1 mRNA or CXCR4 mRNA is administered. In other embodiments, two or more interfering RNAs targeting SDF1 mRNA or CXCR4 mRNA are administered. In further embodiments, interfering RNAs targeting each of SDF1 mRNA and CXCR4 mRNA are administered in combination or in a time interval so as to have overlapping effects.

Knock-down is commonly assessed by measuring the mRNA levels using quantitative polymerase chain reaction (qPCR) amplification or by measuring protein levels by western blot or enzyme-linked immunosorbent assay (ELISA). Analyzing the protein level provides an assessment of both mRNA cleavage as well as translation inhibition. Further techniques for measuring knock-down include RNA solution hybridization, nuclease protection, northern hybridization, gene expression monitoring with a microarray, antibody binding, radioimmunoassay, and fluorescence activated cell analysis.

Inhibition of SDF1 or CXCR4 may also be determined in vitro by evaluating target mRNA levels or target protein levels in, for example, MCF-7 cells (treated with 10 nM 17β-estradiol to induce SDF1 expression) or HUVEC cells, respectively, following transfection of SDF1- or CXCR4-interfering RNA as described infra.

Inhibition of SDF1 mRNA expression or of CXCR4 mRNA expression is also inferred in a human or mammal by observing an improvement in symptoms related to ocular angiogenesis, retinal edema, retinal ischemia, or diabetic retinopathy.

Interfering RNA: In one embodiment of the invention, interfering RNA (e.g., siRNA) has a sense strand and an antisense strand, and the sense and antisense strands comprise a region of at least near-perfect contiguous complementarity of at least 19 nucleotides. In a further embodiment of the invention, interfering RNA (e.g., siRNA) has a sense strand and an antisense strand, and the antisense strand comprises a region of at least near-perfect contiguous complementarity of at least 19 nucleotides to a target sequence of SDF1 mRNA or CXCR4 mRNA, and the sense strand comprises a region of at least near-perfect contiguous identity of at least 19 nucleotides with a target sequence of SDF1 mRNA or CXCR4 mRNA, respectively. In a further embodiment of the invention, the interfering RNA comprises a region of at least 13, 14, 15, 16, 17, or 18 contiguous nucleotides having percentages of sequence complementarity to or, having percentages of sequence identity with, the penultimate 13, 14, 15, 16, 17, or 18 nucleotides, respectively, of the 3′ end of the corresponding target sequence within an mRNA.

The length of each strand of the interfering RNA comprises 19 to 49 nucleotides, and may comprise a length of 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides.

The antisense strand of an siRNA is the active guiding agent of the siRNA in that the antisense strand is incorporated into RISC, thus allowing RISC to identify target mRNAs with at least partial complementarity to the antisense siRNA strand for cleavage or translational repression.

In embodiments of the present invention, interfering RNA target sequences (e.g., siRNA target sequences) within a target mRNA sequence are selected using available design tools. Interfering RNAs corresponding to a SDF1 or CXCR4 target sequence are then tested by transfection of cells expressing the target mRNA followed by assessment of knockdown as described above.

Techniques for selecting target sequences for siRNAs are provided by Tuschl, T. et al., “The siRNA User Guide,” revised May 6, 2004, available on the Rockefeller University web site; by Technical Bulletin #506, “siRNA Design Guidelines,” Ambion Inc. at Ambion's web site; and by other web-based design tools at, for example, the Invitrogen, Dharmacon, Integrated DNA Technologies, Genscript, or Proligo web sites. Initial search parameters can include G/C contents between 35% and 55% and siRNA lengths between 19 and 27 nucleotides. The target sequence may be located in the coding region or in the 5′ or 3′ untranslated regions of the mRNAs.

An embodiment of a 19-nucleotide DNA target sequence for CXCR4 mRNA is present at nucleotides 345 to 363 of SEQ ID NO:4:

| | |
|---|---|
| 5′- ATAACTACACCGAGGAAAT -3′. | SEQ ID NO:6 |

An siRNA of the invention for targeting a corresponding mRNA sequence of SEQ ID NO:6 and having 21-nucleotide strands and a 2-nucleotide 3′ overhang is:

| | |
|---|---|
| 5′- AUAACUACACCGAGGAAAUNN -3′. | SEQ ID NO:7 |
| 3′- NNUAUUGAUGUGGCUCCUUUA -5′. | SEQ ID NO:8 |

Each “N” residue can be any nucleotide (A, C, G, U, T) or modified nucleotide. The 3′ end can have a number of “N” residues between and including 1, 2, 3, 4, 5, and 6. The “N” residues on either strand can be the same residue (e.g., UU, AA, CC, GG, or TT) or they can be different (e.g., AC, AG, AU, CA, CG, CU, GA, GC, GU, UA, UC, or UG). The 3′ overhangs can be the same or they can be different. In one embodiment, both strands have a 3′UU overhang.

An siRNA of the invention for targeting a corresponding mRNA sequence of SEQ ID NO:6 and having 21-nucleotide strands and a 3′UU overhang on each strand is:

| | |
|---|---|
| 5′- AUAACUACACCGAGGAAAUUU -3′ | SEQ ID NO:9 |
| 3′- UUUAUUGAUGUGGCUCCUUUA -5′. | SEQ ID NO:10 |

The interfering RNA may also have a 5′ overhang of nucleotides or it may have blunt ends. An siRNA of the invention for targeting a corresponding mRNA sequence of SEQ ID NO:6 and having 19-nucleotide strands and blunt ends is:

| | |
|---|---|
| 5′- AUAACUACACCGAGGAAAU -3′ | SEQ ID NO:11 |
| 3′- UAUUGAUGUGGCUCCUUUA -5′. | SEQ ID NO:12 |

The strands of a double-stranded interfering RNA (e.g., an siRNA) may be connected to form a hairpin or stem-loop structure (e.g., an shRNA). An shRNA of the invention targeting a corresponding mRNA sequence of SEQ ID NO:6 and having a 19 bp double-stranded stem region and a 3′UU overhang is:

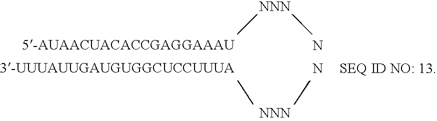

N is a nucleotide A, T, C, G, U, or a modified form known by one of ordinary skill in the art. The number of nucleotides N in the loop is a number between and including 3 to 23, or 5 to 15, or 7 to 13, or 4 to 9, or 9 to 11, or the number of nucleotides N is 9. Some of the nucleotides in the loop can be involved in base-pair interactions with other nucleotides in the loop. Examples of oligonucleotide sequences that can be used to form the loop include 5′-UUCAAGAGA-3′ (Brummelkamp, T. R. et al. (2002) Science 296: 550) and 5′-UUUGUGUAG-3′ (Castanotto, D. et al. (2002) RNA 8:1454). It will be recognized by one of skill in the art that the resulting single chain oligonucleotide forms a stem-loop or hairpin structure comprising a double-stranded region capable of interacting with the RNAi machinery.

The siRNA target sequence identified above can be extended at the 3′ end to facilitate the design of dicer-substrate 27-mer duplexes. Extension of the 19-nucleotide DNA target sequence (SEQ ID NO:6) identified in the CXCR4 DNA sequence (SEQ ID NO:4) by 6 nucleotides yields a 25-nucleotide DNA target sequence present at nucleotides 345 to 369 of SEQ ID NO:4:

| | |
|---|---|
| 5′- ATAACTACACCGAGGAAATGGGCTC -3′. | SEQ ID NO:14 |

A dicer-substrate 27-mer duplex of the invention for targeting a corresponding mRNA sequence of SEQ ID NO:14 is:

| | |
|---|---|
| 5′- AUAACUACACCGAGGAAAUGGGCUC -3′ | SEQ ID NO:15 |
| 3′- UUUAUUGAUGUGGCUCCUUUACCCGAG -5′. | SEQ ID NO:16 |

The two nucleotides at the 3′ end of the sense strand (i.e., the UC nucleotides of SEQ ID NO:15) may be deoxynucleotides for enhanced processing. Design of dicer-substrate 27-mer duplexes from 19-21 nucleotide target sequences, such as provided herein, is further discussed by the Integrated DNA Technologies (IDT) website and by Kim, D.-H. et al., (February, 2005) Nature Biotechnology 23:2; 222-226.

When interfering RNAs are produced by chemical synthesis, phosphorylation at the 5′ position of the nucleotide at the 5′ end of one or both strands (when present) can enhance siRNA efficacy and specificity of the bound RISC complex but is not required since phosphorylation can occur intracellularly.

Table 1 lists examples of SDF1/CXCL12 DNA target sequences of SEQ ID NO:1, SEQ ID NO:2, and SEQ ID NO:3 from which siRNAs of the present invention are designed in a manner as set forth above. SDF1/CXCL12 encodes stromal cell-derived factor 1, as noted above.

TABLE 1
SDF1/CXCL12 Target Sequences for siRNAs

| | | |
|---|---|---|
| SDF1/CXCL12 | # of Starting | |
| Variant 1, 2, and 3 | Nucleotide with | |
| Target Sequences | reference to | |
| in Common | SEQ ID NO:1 | SEQ ID NO: |

| | | |
|---|---|---|
| CGAAAGCCATGTTGCCAGA | 194 | 17 |
| AGACAAGTGTGCATTGACC | 291 | 18 |
| AAAGTGGATTCAGGAGTAC | 317 | 19 |
| GGATTCAGGAGTACCTGGA | 322 | 20 |
| ACGCCAAGGTCGTGGTCGT | 94 | 123 |
| CCAAGGTCGTGGTCGTGCT | 97 | 124 |
| CTACAGATGCCCATGCCGA | 170 | 125 |
| ACAGATGCCCATGCCGATT | 172 | 126 |
| CCATGCCGATTCTTCGAAA | 180 | 127 |
| GCCGATTCTTCGAAAGCCA | 184 | 128 |
| AGCCATGTTGCCAGAGCCA | 198 | 129 |
| TTGCCAGAGCCAACGTCAA | 205 | 130 |
| CCAGAGCCA&CGTCAAGCA | 208 | 131 |
| GAGCCAACGTCAAGCATCT | 211 | 132 |
| CCAACGTCAAGCATCTCAA | 214 | 133 |
| CAACGTCAAGCATCTCAAA | 215 | 134 |
| ACGTCAAGCATCTCAAAAT | 217 | 135 |
| CGTCAAGCATCTCAAAATT | 218 | 136 |
| TCTCAAAATTCTCAACACT | 227 | 137 |
| TCTCAACACTCCAAACTGT | 236 | 138 |
| CAAACTGTGCCCTTCAGAT | 247 | 139 |
| AAACTGTGCCCTTCAGATT | 248 | 140 |
| CGGCTGAAGAACAACAACA | 273 | 141 |
| GCTGAAGAACAACAACAGA | 275 | 142 |
| CTGAAGAACAACAACAGAC | 276 | 143 |
| TGAAGAACAACAACAGACA | 277 | 144 |
| GAAGAACAACAACAGACAA | 278 | 145 |
| AAGAACAACAACAGACAAG | 279 | 146 |
| GAACAACAACAGACAAGTG | 281 | 147 |
| CAACAACAGACAAGTGTGC | 284 | 148 |
| ACAACAGACAAGTGTGCAT | 286 | 149 |
| CAACAGACAAGTGTGCATT | 287 | 150 |
| ACAGACAAGTGTGCATTGA | 289 | 151 |
| AAGTGTGCATTGACCCGAA | 295 | 152 |
| GTGCATTGACCCGAAGCTA | 299 | 153 |
| TGCATTGACCCGAAGCTAA | 300 | 154 |
| GCATTGACCCGAAGCTAAA | 301 | 155 |
| GACCCGAAGCTAAAGTGGA | 306 | 156 |
| ACCCGAAGCTAAAGTGGAT | 307 | 157 |
| CCGAAGCTAAAGTGGATTC | 309 | 158 |
| AAGCTAAAGTGGATTCAGG | 312 | 159 |
| AGCTAAAGTGGATTCAGGA | 313 | 160 |
| GCTAAAGTGGATTCAGGAG | 314 | 161 |
| CTAAAGTGGATTCAGGAGT | 315 | 162 |
| TAAAGTGGATTCAGGAGTA | 316 | 163 |
| AGGAGTACCTGGAGAAAGC | 328 | 164 |
| AGTACCTGGAGAAAGCTTT | 331 | 165 |
| ACCTGGAGAAAGCTTTAAA | 334 | 166 |
| CTGGAGAAAGCTTTAAACA | 336 | 167 |
| TGGAGAAAGCTTTAAACAA | 337 | 168 |
| GGAGAAAGCTTTAAACAAG | 338 | 169 |

| | | |
|---|---|---|
| | # of Starting | |
| SDF1/CXCL12 Variant | Nucleotide with | |
| 1 and 2 Target | reference to | |
| Sequences in Common | SEQ ID NO:1 | SEQ ID NO: |

| | | |
|---|---|---|
| GATTCTTCGAAAGCCATGT | 187 | 21 |
| CAGAGCCAACGTCAAGCAT | 209 | 22 |

| | | |
|---|---|---|
| | # of Starting | |
| SDF1/CxCL12 Variant | Nucleotide with | |
| 1 and 2 Target | reference to | |
| Sequences in Common | SEQ ID NO:1 | SEQ ID NO: |

| | | |
|---|---|---|
| GAGTACCTGGAGAAAGCTT | 330 | 23 |

| | | |
|---|---|---|
| | # of Starting | |
| | Nucleotide with | |
| SDF1/CXCL12 Variant | reference to | |
| 1 Target Sequences | SEQ ID NO:1 | SEQ ID NO: |

| | | |
|---|---|---|
| TGAGGTTTGCCAGCATTTA | 511 | 24 |
| GGTATGATATTGCAGCTTA | 551 | 25 |
| GTTAGTTCCTTCATGATCA | 664 | 26 |
| CCTTCATGATCATCACAAT | 671 | 27 |
| ACAGTCAGGTGGTGGCTTA | 790 | 28 |
| GTCAGGTGGTGGCTTAACA | 793 | 29 |
| CCATGTAGAAGCCACTATT | 970 | 30 |
| CATGTAGAAGCCACTATTA | 971 | 31 |
| AGACAAGTCTTTACAGAAT | 1267 | 32 |
| ATCTGAGAGCTCGCTTTGA | 1301 | 33 |
| GCCATGGAGGCACTAACAA | 1358 | 34 |
| CCATGGAGGCACTAACAAA | 1359 | 35 |
| TCCGAAATCAGAAGCGAAA | 1388 | 36 |
| GGTGACATTTCCATGCATA | 1492 | 37 |
| GTGACATTTCCATGCATAA | 1493 | 38 |
| GACATTTCCATGCATAAAT | 1495 | 39 |
| TATTTGAAGTGGAGCCATA | 1653 | 40 |
| ATCTCAAACTACTGGCAAT | 1701 | 41 |
| ACTACTGGCAATTTGTAAA | 1708 | 42 |
| GCTAATTGTTTCATGGTAT | 1842 | 43 |
| GTATAAACGTCCTACTGTA | 1857 | 44 |
| TATAAACGTCCTACTGTAT | 1858 | 45 |
| TAAACGTCCTACTGTATGT | 1860 | 46 |
| TGTTAGTGATGGAGCTTAA | 1913 | 47 |
| GTTAGTGATGGAGCTTAAA | 1914 | 48 |

| | | |
|---|---|---|
| | # of Starting | |
| | Nucleotide with | |
| SDF1/CXCL12 Variant | reference to | |
| 2 Target Sequences | SEQ ID NO:2 | SEQ ID NO: |

| | | |
|---|---|---|
| TTGCACACTTTGCCATATT | 494 | 49 |
| TGCACACTTTGCCATATTT | 495 | 50 |
| CTTAGACTAAGGCCATTAT | 598 | 51 |
| TCCACGTTCTGCTCATCAT | 996 | 52 |
| CCACGTTCTGCTCATCATT | 997 | 53 |
| ACGTTCTGCTCATCATTCT | 999 | 54 |
| AAACAAGCATTCACAACTT | 1506 | 55 |
| ACAGGACATTTCTCTAAGA | 1695 | 56 |
| AGTCGATAGAGCTGTATTA | 1783 | 57 |
| GTCGATAGAGCTGTATTAA | 1784 | 58 |
| AGCTGTATTAAGCCACTTA | 1792 | 59 |
| TCATTCAGTCTTACGAATA | 1862 | 60 |
| TGATTAAAGACTCCAGTTA | 1892 | 61 |
| GATTAAAGACTCCAGTTAA | 1893 | 62 |
| ATTTCAAATTGGAGCTTCA | 2081 | 63 |
| TCAATCCAGCTATGTTATA | 2155 | 64 |
| CTCTCACTATACCAGTATA | 2249 | 65 |
| GGCAGTCATTATCCAGGTA | 2284 | 66 |
| CCTAAGCAGACCACTGATA | 3225 | 67 |
| GAGAAGGCCAATTCCTATA | 3386 | 68 |
| CTATACGCAGCGTGCTTTA | 3400 | 69 |
| TATACGCAGCGTGCTTTAA | 3401 | 70 |
| GAAACAACTCTTTGAGAAA | 3431 | 71 |

| | | |
|---|---|---|
| | # of Starting | |
| | Nucleotide with | |
| SDF1/CXCL12 Variant | reference to | |
| 3 Target Sequences | SEQ ID NO:3 | SEQ ID NO: |

| | | |
|---|---|---|
| TAGTTATCTGCCACCTCGA | 447 | 72 |
| GTTATCTGCCACCTCGAGA | 449 | 73 |
| TTATCTGCCACCTCGAGAT | 450 | 74 |

Table 2 lists examples of CXCR4 DNA target sequences of SEQ ID NO:4 and SEQ ID NO:5 from which siRNAs of the present invention are designed in a manner as set forth above. CXCR4 encodes chemokine (C—X—C motif) receptor 4, the receptor for SDF1 as noted above.

TABLE 2
CXCR4 Target Sequences for siRNAs

| | | |
|---|---|---|
| | # of Starting | |
| CXCR4 variant 1 and 2 | Nucleotide with | |
| Target Sequences | reference to | |
| in Common | SEQ ID NO:4 | SEQ ID NO: |

| | | |
|---|---|---|
| ATAACTACACCGAGGAAAT | 345 | 6 |
| TCTTCTTAACTGGCATTGT | 459 | 75 |
| GATTGGTCATCCTGGTCAT | 486 | 76 |
| CTGAGAAGCATGACGGACA | 521 | 77 |
| TGAGAAGCATGACGGACAA | 522 | 78 |
| GAAGCATGACGGACAAGTA | 525 | 79 |
| CAGTTGATGCCGTGGCAAA | 600 | 80 |
| TATGCAAGGCAGTCCATGT | 639 | 81 |
| GCAGTCCATGTCATCTACA | 647 | 82 |
| CATCTTTGCCAACGTCAGT | 832 | 83 |
| ATATCTGTGACCGCTTCTA | 867 | 84 |
| CATCATGGTTGGCCTTATC | 925 | 85 |
| CTGTCCTGCTATTGCATTA | 962 | 86 |
| TGTCCTGCTATTGCATTAT | 963 | 87 |
| TATCATCTCCAAGCTGTCA | 979 | 88 |
| GTTGGCTGCCTTACTACAT | 1068 | 89 |
| TTCTTCCACTGTTGTCTGA | 1190 | 90 |
| TCTTCCACTGTTGTCTGAA | 1191 | 91 |
| ATCTGTTTCCACTGAGTCT | 1330 | 92 |
| GACTGACCAATATTGTACA | 1448 | 93 |
| ATTGATGTGTGTCTAGGCA | 1557 | 94 |
| GTCTCGTGGTAGGACTGTA | 1607 | 95 |
| CTCGTGGTAGGACTGTAGA | 1609 | 96 |
| TCGTGGTAGGACTGTAGAA | 1610 | 97 |
| GAACATTCCAGAGCGTGTA | 1638 | 98 |
| GTGTAGTGAATCACGTAAA | 1652 | 99 |
| TGAATCACGTAAAGCTAGA | 1658 | 100 |
| GAATCACGTAAAGCTAGAA | 1659 | 101 |
| ATCACGTAAAGCTAGAAAT | 1661 | 102 |
| TCACGTAAAGCTAGAAATG | 1662 | 103 |
| CACGTAAAGCTAGAAATGA | 1663 | 104 |
| ACGTAAAGCTAGAAATGAT | 1664 | 105 |
| TCCTGTTCTTAAGACGTGA | 1732 | 106 |
| CCTGTTCTTAAGACGTGAT | 1733 | 107 |
| CTGTAGAAGATGGCACTTA | 1756 | 108 |
| TGTAGAAGATGGCACTTAT | 1757 | 109 |
| GTAGAAGATGGCACTTATA | 1758 | 110 |
| TAGAAGATGGCACTTATAA | 1759 | 111 |
| GATGGCACTTATAACCAAA | 1764 | 112 |
| ATGGATTGGTCATCCTGGT | 483 | 113 |
| GCTATTGCATTATCATCTC | 969 | 114 |
| CCAAGCTGTCACACTCCAA | 987 | 115 |
| AGCACGCACTCACCTCTGT | 1257 | 116 |
| TGCTGTATGTCTCGTGGTA | 1599 | 117 |
| TGTAGTGAATCACGTAAAG | 1653 | 118 |
| AGATAACTACACCGAGGAA | 122 | 170 |
| GATAACTACACCGAGGAAA | 123 | 171 |
| GGAAATGGGCTCAGGGGAC | 137 | 172 |
| GGGACTATGACTCCATGAA | 151 | 173 |
| CCATGAAGGAACCCTGTTT | 163 | 174 |
| CCGTGAAGAAAATGCTAAT | 182 | 175 |
| TAACTGGCATTGTGGGCAA | 244 | 176 |
| TCATCCTGGTCATGGGTTA | 271 | 177 |
| GGTCATGGGTTACCAGAAG | 278 | 178 |
| TCATGGGTTACCAGAAGAA | 280 | 179 |
| CAGAAGAAACTGAGAAGCA | 291 | 180 |
| AGAAGAAACTGAGAAGCAT | 292 | 181 |
| GAAGAAACTGAGAAGCATG | 293 | 182 |
| AAGAAACTGAGAAGCATGA | 294 | 183 |
| GCAAGGCAGTCCATGTCAT | 421 | 184 |
| AGGCAGTCCATGTCATCTA | 424 | 185 |
| TCATCAGTCTGGACCGCTA | 481 | 186 |
| ACCAACAGTCAGAGGCCAA | 519 | 187 |
| CAAGGAAGCTGTTGGCTGA | 535 | 188 |
| AGGAAGCTGTTGGCTGAAA | 537 | 189 |
| TGGCTGAAAAGGTGGTCTA | 547 | 190 |
| CTGAAAAGGTGGTCTATGT | 550 | 191 |
| CCGACTTCATCTTTGCCAA | 604 | 192 |
| CCAACGTCAGTGAGGCAGA | 619 | 193 |
| CAGTGAGGCAGATGACAGA | 626 | 194 |
| GTGAGGCAGATGACAGATA | 628 | 195 |
| GAGGCAGATGACAGATATA | 630 | 196 |
| GCACATCATGGTTGGCCTT | 701 | 197 |
| TGTCACACTCCAAGGGCCA | 772 | 198 |
| ACTCCAAGGGCCACCAGAA | 778 | 199 |
| CCTCAAGACCACAGTCATC | 806 | 200 |
| CCTTCATCCTCCTGGAAAT | 883 | 201 |
| CCTGGAAATCATCAAGCAA | 893 | 202 |
| CAAGCAAGGGTGTGAGTTT | 905 | 203 |
| GGGTGTGAGTTTGAGAACA | 912 | 204 |
| TTGAGAACACTGTGCACAA | 922 | 205 |
| GGTCCAGCCTCAAGATCCT | 1063 | 206 |
| CCTCAAGATCCTCTCCAAA | 1070 | 207 |
| CAAAGGAAAGCGAGGTGGA | 1085 | 208 |
| AGGAAAGCGAGGTGGACAT | 1088 | 209 |
| AAAGCGAGGTGGACATTCA | 1091 | 210 |
| CCACTGAGTCTGAGTCTTC | 1117 | 211 |
| GAGTCTGAGTCTTCAAGTT | 1122 | 212 |
| AGTCTGAGTCTTCAAGTTT | 1123 | 213 |

| | | |
|---|---|---|
| | # of Starting | |
| | Nucleotide with | |
| CXCR4 variant 1 | reference to | |
| Target Sequences | SEQ ID NO:4 | SEQ ID NO: |

| | | |
|---|---|---|
| GGAGAGTTGTAGGATTCTA | 159 | 119 |
| GTTGTAGGATTCTACATTA | 164 | 120 |
| TGCTGAATTGGAAGTGAAT | 288 | 121 |

| | | |
|---|---|---|
| | # of Starting | |
| | Nucleotide with | |
| CXCR4 variant 2 | reference to | |
| Target Sequence | SEQ ID NO:5 | SEQ ID NO: |

| | | |
|---|---|---|
| TCAGTATATACACTTCAGA | 106 | 122 |

| | | |
|---|---|---|
| | # of Starting | |
| | Nucleotide with | |
| CXCR4 variant 1 | reference to | |
| Target Sequences | SEQ ID NO:4 | SEQ ID NO: |

| | | |
|---|---|---|
| GGAGAGTTGTAGGATTCTA | 159 | 119 |
| GTTGTAGGATTCTACATTA | 164 | 120 |
| TGCTGAATTGGAAGTGAAT | 288 | 121 |

| | | |
|---|---|---|
| | # of Starting | |
| | Nucleotide with | |
| CXCR4 variant 1 | reference to | |
| Target Sequences | SEQ ID NO:5 | SEQ ID NO: |

| | | |
|---|---|---|
| TCAGTATATACACTTCAGA | 106 | 122 |

As cited in the examples above, one of skill in the art is able to use the target sequence information provided in Tables 1 or 2 to design interfering RNAs having a length shorter or longer than the sequences provided in the tables and by referring to the sequence position in any of SEQ ID NO:1-SEQ ID NO:5 and adding or deleting nucleotides complementary or near complementary to any of SEQ ID NO:1-SEQ ID NO:5, respectively.

The target RNA cleavage reaction guided by siRNAs and other forms of interfering RNA is highly sequence specific. In general, siRNA containing a sense nucleotide strand identical in sequence to a portion of the target mRNA and an antisense nucleotide strand exactly complementary to a portion of the target mRNA are siRNA embodiments for inhibition of mRNAs cited herein. However, 100% sequence complementarity between the antisense siRNA strand and the target mRNA, or between the antisense siRNA strand and the sense siRNA strand, is not required to practice the present invention. Thus, for example, the invention allows for sequence variations that might be expected due to genetic mutation, strain polymorphism, or evolutionary divergence.

In one embodiment of the invention, the antisense strand of the siRNA has at least near-perfect contiguous complementarity of at least 19 nucleotides with the target mRNA.

The relationship between a target mRNA (sense strand) and one strand of an siRNA (the sense strand) is that of identity. The sense strand of an siRNA is also called a passenger strand, if present. The relationship between a target mRNA (sense strand) and the other strand of an siRNA (the antisense strand) is that of complementarity. The antisense strand of an siRNA is also called a guide strand.

The penultimate base in a nucleic acid sequence that is written in a 5′ to 3′ direction is the next to the last base, i.e., the base next to the 3′ base. The penultimate 13 bases of a nucleic acid sequence written in a 5′ to 3′ direction are the last 13 bases of a sequence next to the 3′ base and not including the 3′ base. Similarly, the penultimate 14, 15, 16, 17, or 18 bases of a nucleic acid sequence written in a 5′ to 3′ direction are the last 14, 15, 16, 17, or 18 bases of a sequence, respectively, next to the 3′ base and not including the 3′ base.

The phrase “a region of at least 13 contiguous nucleotides having at least 90% sequence complementarity to, or at least 90% sequence identity with, the penultimate 13 nucleotides of the 3′ end of an mRNA corresponding to any one of (a sequence identifier)” allows a one nucleotide substitution. Two nucleotide substitutions (i.e., 11/13=85% identity/complementarity) are not included in such a phrase.

In one embodiment of the invention, the region of contiguous nucleotides is a region of at least 14 contiguous nucleotides having at least 85% sequence complementarity to, or at least 85% sequence identity with, the penultimate 14 nucleotides of the 3′ end of an mRNA corresponding to the sequence identified by each sequence identifier. Two nucleotide substitutions (i.e., 12/14=86% identity/complementarity) are included in such a phrase.

In a further embodiment of the invention, the region of contiguous nucleotides is a region of at least 15, 16, 17, or 18 contiguous nucleotides having at least 80% sequence complementarity to, or at least 80% sequence identity with, the penultimate 14 nucleotides of the 3′ end of an mRNA corresponding to the sequence of the sequence identifier. Three nucleotide substitutions are included in such a phrase.

The target sequence in the mRNAs corresponding to SEQ ID NO:1-SEQ ID NO:5 may be in the 5′ or 3′ untranslated regions of the mRNA as well as in the coding region of the mRNA.

One or both of the strands of double-stranded interfering RNA may have a 3′ overhang of from 1 to 6 nucleotides, which may be ribonucleotides or deoxyribonucleotides or a mixture thereof. The nucleotides of the overhang are not base-paired. In one embodiment of the invention, the interfering RNA comprises a 3′ overhang of TT or UU. In another embodiment of the invention, the interfering RNA comprises at least one blunt end. The termini usually have a 5′ phosphate group or a 3′ hydroxyl group. In other embodiments, the antisense strand has a 5′ phosphate group, and the sense strand has a 5′ hydroxyl group. In still other embodiments, the termini are further modified by covalent addition of other molecules or functional groups.

The sense and antisense strands of the double-stranded siRNA may be in a duplex formation of two single strands as described above or may be a single molecule where the regions of complementarity are base-paired and are covalently linked by a hairpin loop so as to form a single strand. It is believed that the hairpin is cleaved intracellularly by a protein termed dicer to form an interfering RNA of two individual base-paired RNA molecules.

Interfering RNAs may differ from naturally-occurring RNA by the addition, deletion, substitution or modification of one or more nucleotides. Non-nucleotide material may be bound to the interfering RNA, either at the 5′ end, the 3′ end, or internally. Such modifications are commonly designed to increase the nuclease resistance of the interfering RNAs, to improve cellular uptake, to enhance cellular targeting, to assist in tracing the interfering RNA, to further improve stability, or to reduce the potential for activation of the interferon pathway. For example, interfering RNAs may comprise a purine nucleotide at the ends of overhangs. Conjugation of cholesterol to the 3′ end of the sense strand of an siRNA molecule by means of a pyrrolidine linker, for example, also provides stability to an siRNA.

Further modifications include a 3′ terminal biotin molecule, a peptide known to have cell-penetrating properties, a nanoparticle, a peptidomimetic, a fluorescent dye, or a dendrimer, for example.

Nucleotides may be modified on their base portion, on their sugar portion, or on the phosphate portion of the molecule and function in embodiments of the present invention. Modifications include substitutions with alkyl, alkoxy, amino, deaza, halo, hydroxyl, thiol groups, or a combination thereof, for example. Nucleotides may be substituted with analogs with greater stability such as replacing a ribonucleotide with a deoxyribonucleotide, or having sugar modifications such as 2′ OH groups replaced by 2′ amino groups, 2′ O-methyl groups, 2′ methoxyethyl groups, or a 2′-O, 4′-C methylene bridge, for example. Examples of a purine or pyrimidine analog of nucleotides include a xanthine, a hypoxanthine, an azapurine, a methylthioadenine, 7-deaza-adenosine and O- and N-modified nucleotides. The phosphate group of the nucleotide may be modified by substituting one or more of the oxygens of the phosphate group with nitrogen or with sulfur (phosphorothioates). Modifications are useful, for example, to enhance function, to improve stability or permeability, or to direct localization or targeting.

There may be a region or regions of the antisense interfering RNA strand that is (are) not complementary to a portion of SEQ ID NO:1-SEQ ID NO:5. Non-complementary regions may be at the 3′, 5′ or both ends of a complementary region or between two complementary regions.

Interfering RNAs may be generated exogenously by chemical synthesis, by in vitro transcription, or by cleavage of longer double-stranded RNA with dicer or another appropriate nuclease with similar activity. Chemically synthesized interfering RNAs, produced from protected ribonucleoside phosphoramidites using a conventional DNA/RNA synthesizer, may be obtained from commercial suppliers such as Ambion Inc. (Austin, Tex.), Invitrogen (Carlsbad, Calif.), or Dharmacon (Lafayette, Colo.). Interfering RNAs are purified by extraction with a solvent or resin, precipitation, electrophoresis, chromatography, or a combination thereof, for example. Alternatively, interfering RNA may be used with little if any purification to avoid losses due to sample processing.

Interfering RNAs can also be expressed endogenously from plasmid or viral expression vectors or from minimal expression cassettes, for example, PCR generated fragments comprising one or more promoters and an appropriate template or templates for the interfering RNA. Examples of commercially available plasmid-based expression vectors for shRNA include members of the pSilencer series (Ambion, Austin, Tex.) and pCpG-siRNA (InvivoGen, San Diego, Calif.). Viral vectors for expression of interfering RNA may be derived from a variety of viruses including adenovirus, adeno-associated virus, lentivirus (e.g., HIV, FIV, and EIAV), and herpes virus. Examples of commercially available viral vectors for shRNA expression include pSilencer adeno (Ambion, Austin, Tex.) and pLenti6/BLOCK-iT™-DEST (Invitrogen, Carlsbad, Calif.). Selection of viral vectors, methods for expressing the interfering RNA from the vector and methods of delivering the viral vector are within the ordinary skill of one in the art. Examples of kits for production of PCR-generated shRNA expression cassettes include Silencer Express (Ambion, Austin, Tex.) and siXpress (Mirus, Madison, Wis.). A first interfering RNA may be administered via in vivo expression from a first expression vector capable of expressing the first interfering RNA and a second interfering RNA may be administered via in vivo expression from a second expression vector capable of expressing the second interfering RNA, or both interfering RNAs may be administered via in vivo expression from a single expression vector capable of expressing both interfering RNAs.

Interfering RNAs may be expressed from a variety of eukaryotic promoters known to those of ordinary skill in the art, including pol III promoters, such as the U6 or H1 promoters, or pol II promoters, such as the cytomegalovirus promoter. Those of skill in the art will recognize that these promoters can also be adapted to allow inducible expression of the interfering RNA.

Hybridization under Physiological Conditions: In certain embodiments of the present invention, an antisense strand of an interfering RNA hybridizes with an mRNA in vivo as part of the RISC complex.

The above-described in vitro hybridization assay provides a method of predicting whether binding between a candidate siRNA and a target will have specificity. However, in the context of the RISC complex, specific cleavage of a target can also occur with an antisense strand that does not demonstrate high stringency for hybridization in vitro.

Single-stranded interfering RNA: As cited above, interfering RNAs ultimately function as single strands. Single-stranded (ss) interfering RNA has been found to effect mRNA silencing, albeit less efficiently than double-stranded RNA. Therefore, embodiments of the present invention also provide for administration of a ss interfering RNA that hybridizes under physiological conditions to a portion of any of SEQ ID NO:1-SEQ ID NO:5 and has a region of at least near-perfect contiguous complementarity of at least 19 nucleotides with the hybridizing portion of SEQ ID NO:1-SEQ ID NO:5, respectively. The ss interfering RNA of Table 1 or Table 2 has a length of 19 to 49 nucleotides as for the ds interfering RNA cited above. The ss interfering RNA has a 5′ phosphate or is phosphorylated in situ or in vivo at the 5′ position. The term “5′ phosphorylated” is used to describe, for example, polynucleotides or oligonucleotides having a phosphate group attached via ester linkage to the C5 hydroxyl of the sugar (e.g., ribose, deoxyribose, or an analog of same) at the 5′ end of the polynucleotide or oligonucleotide.

SS interfering RNAs are synthesized chemically or by in vitro transcription or expressed endogenously from vectors or expression cassettes as for ds interfering RNAs. 5′ Phosphate groups may be added via a kinase, or a 5′ phosphate may be the result of nuclease cleavage of an RNA. Delivery is as for ds interfering RNAs. In one embodiment, ss interfering RNAs having protected ends and nuclease resistant modifications are administered for silencing. SS interfering RNAs may be dried for storage or dissolved in an aqueous solution. The solution may contain buffers or salts to inhibit annealing or for stabilization.

Hairpin interfering RNA: A hairpin interfering RNA is a single molecule (e.g., a single oligonucleotide chain) that comprises both the sense and antisense strands of an interfering RNA in a stem-loop or hairpin structure (e.g., a shRNA). For example, shRNAs can be expressed from DNA vectors in which the DNA oligonucleotides encoding a sense interfering RNA strand are linked to the DNA oligonucleotides encoding the reverse complementary antisense interfering RNA strand by a short spacer. If needed for the chosen expression vector, 3′ terminal T's and nucleotides forming restriction sites may be added. The resulting RNA transcript folds back onto itself to form a stem-loop structure.

Mode of administration: Interfering RNA may be delivered via aerosol, buccal, dermal, intradermal, inhaling, intramuscular, intranasal, intraocular, intrapulmonary, intravenous, intraperitoneal, nasal, ocular, oral, otic, parenteral, patch, subcutaneous, sublingual, topical, or transdermal administration, for example. Further forms of a medicament of various embodiments of the present invention are capable of also taking the form of a tablet, pill, capsule, and/or the like.

Administration may be directly to the eye by ocular tissue administration such as periocular, conjunctival, subtenon, intracameral, intravitreal, intraocular, subretinal, subconjunctival, retrobulbar, intracanalicular, or suprachoroidal administration; by injection, by direct application to the eye using a catheter or other placement device such as a retinal pellet, intraocular insert, suppository or an implant comprising a porous, non-porous, or gelatinous material; by topical ocular drops or ointments; or by a slow release device in the cul-de-sac or implanted adjacent to the sclera (transscleral) or within the eye. Intracameral injection may be through the cornea into the anterior chamber to allow the agent to reach the trabecular meshwork. Intracanalicular injection may be into the venous collector channels draining Schlemm's canal or into Schlemm's canal.

Administration may be directly to the ear via, for example, topical otic drops or ointments, slow release devices in the ear or implanted adjacent to the ear. Local administration includes otic intramuscular, intratympanic cavity and intracochlear injection routes of administration. Furthermore, agents can be administered to the inner ear by placement of a gelfoam, or similar absorbent and adherent product, soaked with the interfering RNA against the window membrane of the middle/inner ear or adjacent structure.

Administration may be directly to the lungs, via, for example, an aerosolized preparation, and by inhalation via an inhaler or a nebulizer, for example

Subject: A subject in need of treatment for a NV-related condition or at risk for developing a NV-related condition is a human or other mammal having a NV-related condition or at risk of developing a NV-related condition. A NV-related condition includes, for example, ocular NV, abnormal angiogenesis, retinal vascular permeability, retinal edema, diabetic retinopathy particularly proliferative diabetic retinopathy, diabetic macular edema, exudative age-related macular degeneration, sequela associated with retinal ischemia, and posterior segment NV, for example, associated with undesired or inappropriate activity of SDF1 or CXCR4 as cited herein.

Ocular structures associated with a NV-related condition may include the eye, cornea, trabecular meshwork, iris, ciliary body, lens, retina, choroid, optic nerve, optic nerve head, sclera, anterior or posterior segments, for example.

Otic structures associated with such disorders may include the inner ear, middle ear, outer ear, tympanic cavity or membrane, cochlea, or Eustachian tube, for example.

Pulmonary structures associated with such disorders may include the nose, mouth, pharynx, larynx, bronchial tubes, trachea, carina (the ridge separating the opening of the right and left main bronchi), and lungs, particularly the lower lungs, such as bronchioli and alveoli.

A subject may also be an otic cell, a lung cell, an ocular cell, cell culture, organ or an ex vivo organ or tissue.

Formulations and Dosage: Pharmaceutical formulations comprise interfering RNAs, or salts thereof, of the invention up to 99% by weight mixed with a physiologically acceptable carrier medium such as water, buffer, saline, glycine, hyaluronic acid, mannitol, and the like.

Interfering RNAs of the present invention are administered as solutions, suspensions, or emulsions. The following are examples of possible formulations embodied by this invention.

| | Amount in weight % |
|---|---|
| Interfering RNA | up to 99; 0.1-99; 0.1-50; 0.5-10.0 |
| Hydroxypropylmethylcellulose | 0.5 |
| Sodium chloride | 0.8 |
| Benzalkonium Chloride | 0.01 |
| EDTA | 0.01 |
| NaOH/HCl | Qs pH 7.4 |
| Purified water (RNase-free) | Qs 100 Ml |
| Interfering RNA | up to 99; 0.1-99; 0.1-50; 0.5-10.0 |
| Phosphate Buffered Saline | 1.0 |
| Benzalkonium Chloride | 0.01 |
| Polysorbate 80 | 0.5 |
| Purified water (RNase-free) | q.s. to 100% |
| Interfering RNA | up to 99; 0.1-99; 0.1-50; 0.5-10.0 |
| Monobasic sodium phosphate | 0.05 |
| Dibasic sodium phosphate | 0.15 |
| (anhydrous) | |
| Sodium chloride | 0.75 |
| Disodium EDTA | 0.05 |
| Cremophor EL | 0.1 |
| Benzalkonium chloride | 0.01 |
| HCl and/or NaOH | pH 7.3-7.4 |
| Purified water (RNase-free) | q.s. to 100% |
| Interfering RNA | up to 99; 0.1-99; 0.1-50; 0.5-10.0 |
| Phosphate Buffered Saline | 1.0 |
| Hydroxypropyl-β-cyclodextrin | 4.0 |
| Purified water (RNase-free) | q.s. to 100% |

Generally, an effective amount of the interfering RNAs of embodiments of the invention results in an extracellular concentration at the surface of the target cell of from 100 pM to 1000 nM, or from 1 nM to 400 nM, or from 5 nM to about 100 nM, or about 10 nM. The dose required to achieve this local concentration will vary depending on a number of factors including the delivery method, the site of delivery, the number of cell layers between the delivery site and the target cell or tissue, whether delivery is local or systemic, etc. The concentration at the delivery site may be considerably higher than it is at the surface of the target cell or tissue. Topical compositions are delivered to the surface of the target organ one to four times per day, or on an extended delivery schedule such as daily, weekly, bi-weekly, monthly, or longer, according to the routine discretion of a skilled clinician. The pH of the formulation is about pH 4-9, or pH 4.5 to pH 7.4.

Therapeutic treatment of patients with siRNAs directed against SDF1 mRNA or CXCR4 mRNA is expected to be beneficial over small molecule treatments by increasing the duration of action, thereby allowing less frequent dosing and greater patient compliance.

An effective amount of a formulation may depend on factors such as the age, race, and sex of the subject, the severity of the NV-related condition, the rate of target gene transcript/protein turnover, the interfering RNA potency, and the interfering RNA stability, for example. In one embodiment, the interfering RNA is delivered topically to a target organ and reaches SDF1 mRNA- or CXCR4 mRNA-containing tissue at a therapeutic dose thereby ameliorating a NV-related process.

Acceptable carriers: An acceptable carrier refers to those carriers that cause at most, little to no ocular irritation, provide suitable preservation if needed, and deliver one or more interfering RNAs of the present invention in a homogenous dosage. An acceptable carrier for administration of interfering RNA of embodiments of the present invention include the cationic lipid-based transfection reagents TransIT®-TKO (Mirus Corporation, Madison, Wis.), LIPOFECTIN®, Lipofectamine, OLIGOFECTAMINE™ (Invitrogen, Carlsbad, Calif.), or DHARMAFECT™ (Dharmacon, Lafayette, Colo.); polycations such as polyethyleneimine; cationic peptides such as Tat, polyarginine, or Penetratin (Antp peptide); or liposomes. Liposomes are formed from standard vesicle-forming lipids and a sterol, such as cholesterol, and may include a targeting molecule such as a monoclonal antibody having binding affinity for endothelial cell surface antigens, for example. Further, the liposomes may be PEGylated liposomes.

The interfering RNAs may be delivered in solution, in suspension, or in bioerodible or non-bioerodible delivery devices. The interfering RNAs can be delivered alone or as components of defined, covalent conjugates. The interfering RNAs can also be complexed with cationic lipids, cationic peptides, or cationic polymers; complexed with proteins, fusion proteins, or protein domains with nucleic acid binding properties (e.g., protamine); or encapsulated in nanoparticles. Tissue- or cell-specific delivery can be accomplished by the inclusion of an appropriate targeting moiety such as an antibody or antibody fragment.

For ophthalmic, otic, or pulmonary delivery, an interfering RNA may be combined with opthalmologically, optically, or pulmonary acceptable preservatives, co-solvents, surfactants, viscosity enhancers, penetration enhancers, buffers, sodium chloride, or water to form an aqueous, sterile suspension or solution. Solution formulations may be prepared by dissolving the interfering RNA in a physiologically acceptable isotonic aqueous buffer. Further, the solutions may include an acceptable surfactant to assist in dissolving the inhibitor. Viscosity building agents, such as hydroxymethyl cellulose, hydroxyethyl cellulose, methylcellulose, polyvinylpyrrolidone, or the like may be added to the compositions of the present invention to improve the retention of the compound.

In order to prepare a sterile ointment formulation, the interfering RNA is combined with a preservative in an appropriate vehicle, such as mineral oil, liquid lanolin, or white petrolatum. Sterile gel formulations may be prepared by suspending the interfering RNA in a hydrophilic base prepared from the combination of, for example, CARBOPOL-940 (BF Goodrich, Charlotte, N.C.), or the like, according to methods known in the art. VISCOAT® (Alcon Laboratories, Inc., Fort Worth, Tex.) may be used for intraocular injection, for example. Other compositions of the present invention may contain penetration enhancing agents such as cremephor and TWEEN® 80 (polyoxyethylene sorbitan monolaureate, Sigma Aldrich, St. Louis, Mo.), in the event the interfering RNA is less penetrating in the organ or tissue of interest.

Kits: Embodiments of the present invention provide a kit that includes reagents for attenuating the expression of an mRNA as cited herein in a cell. The kit contains an siRNA or an shRNA expression vector. For siRNAs and non-viral shRNA expression vectors the kit also may contain a transfection reagent or other suitable delivery vehicle. For viral shRNA expression vectors, the kit may contain the viral vector and/or the necessary components for viral vector production (e.g., a packaging cell line as well as a vector comprising the viral vector template and additional helper vectors for packaging). The kit may also contain positive and negative control siRNAs or shRNA expression vectors (e.g., a non-targeting control siRNA or an siRNA that targets an unrelated mRNA). The kit also may contain reagents for assessing knockdown of the intended target gene (e.g., primers and probes for quantitative PCR to detect the target mRNA and/or antibodies against the corresponding protein for western blots). Alternatively, the kit may comprise an siRNA sequence or an shRNA sequence and the instructions and materials necessary to generate the siRNA by in vitro transcription or to construct an shRNA expression vector.

A pharmaceutical combination in kit form is further provided that includes, in packaged combination, a carrier means adapted to receive a container means in close confinement therewith and a first container means including an interfering RNA composition and an acceptable carrier. Such kits can further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, can also be included in the kit.

The ability of interfering RNA to knock-down the levels of endogenous target gene expression in, for example, human umbilical vein endothelial cells (HUVEC cells) is evaluated in vitro as follows. HUVEC cells (Cambrex, East Rutherford, N.J.), are plated 24-48 h prior to transfection in EBM-2 Medium (Cambrex). Transfection is performed using LipofectAMINE (Invitrogen, Carlsbad, Calif.) according to the manufacturer's instructions at interfering RNA (e.g., siRNA) concentrations ranging from 0.1 nM-100 nM. siCONTROL™ Non-Targeting siRNA #1 and siCONTROL™ Cyclophilin B siRNA (Dharmacon) are used as negative and positive controls, respectively. Target mRNA levels and cyclophilin B mRNA (PPIB, NM_{—}000942) levels are assessed by qPCR 24 h post-transfection using, for example, TAQMAN® forward and reverse primers and a probe set that preferably encompasses the target site (Applied Biosystems, Foster City, Calif.). The positive control siRNA gives essentially complete knockdown of cyclophilin B mRNA when transfection efficiency is 100%. Therefore, target mRNA knockdown is corrected for transfection efficiency by reference to the cyclophilin B mRNA level in HUVEC cells transfected with the cyclophilin B siRNA. Target protein levels may be assessed approximately 72 h post-transfection (actual time dependent on protein turnover rate) by western blot, for example. Standard techniques for RNA and/or protein isolation from cultured cells are well-known to those skilled in the art. To reduce the chance of non-specific, off-target effects, the lowest possible concentration of interfering RNA is used that produces the desired level of knock-down in target gene expression.

The references cited herein, to the extent that they provide exemplary procedural or other details supplementary to those set forth herein, are specifically incorporated by reference.

Those of skill in the art, in light of the present disclosure, will appreciate that obvious modifications of the embodiments disclosed herein can be made without departing from the spirit and scope of the invention. All of the embodiments disclosed herein can be made and executed without undue experimentation in light of the present disclosure. The full scope of the invention is set out in the disclosure and equivalent embodiments thereof. The specification should not be construed to unduly narrow the full scope of protection to which the present invention is entitled.

While a particular embodiment of the invention has been shown and described, numerous variations and alternate embodiments will occur to those skilled in the art. Accordingly, it is intended that the invention be limited only in terms of the appended claims.

The invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes to the claims that come within the meaning and range of equivalency of the claims are to be embraced within their scope. Further, all published documents, patents, and applications mentioned herein are hereby incorporated by reference, as if presented in their entirety.

### PROPHETIC EXAMPLE 1

### Theoretical Data

### Interfering RNA for Specifically Silencing CXCR4 in MDA-MB-231 Cells

Transfection of MDA-MB-231 cells (ATCC, Manassas, Va.) is accomplished using standard in vitro concentrations (0.1-100 nM) of CXCR4 siRNAs or siCONTROL Non-targeting siRNA #2 (NTC2) and LipofectAMINE 2000 transfection reagent (Invitrogen, Carlsbad, Calif.). All siRNAs are dissolved in 1×siRNA buffer, an aqueous solution of 20 mM KCl, 6 mM HEPES (pH 7.5), 0.2 mM MgCl₂. Control samples include a buffer control in which the volume of siRNA is replaced with an equal volume of 1×siRNA buffer (Null). CXCR4 mRNA level is determined by qRT-PCR using Assays-On-Demand Gene Expression kits, TaqMan Universal PCR Master Mix, and an ABI PRISM 7700 Sequence Detector (Applied Biosystems, Foster City, Calif.). CXCR4 mRNA expression is normalized to PPIB (cyclophilin B) mRNA level, and is reported relative to CXCR4 expression in non-transfected cells. The CXCR4 siRNAs are double-stranded interfering RNAs having specificity for 19-nucleotide sequences contained within the CXCR4 mRNA sequence (derived from SEQ ID NO:4 and SEQ ID NO:5). It is expected that the siRNAs will cause about at least a 70% reduction in CXCR4 expression at 100 mM. Some of the siRNAs may lose efficacy at 10 nM and below. Some of the siRNAs will maintain efficacy at 10 nM, but will lose efficacy at 1 nM and below. Some of the siRNAs will maintain efficacy at 1 nM, and cause significant reduction in CXCR4 expression at 0.1 nM.

### PROPHETIC EXAMPLE 2

### Theoretical Data

### Interfering RNA for Specifically Silencing SDF1 in HUVEC Cells

Transfection of HUVEC cells (Cambrex, East Rutherford, N.J.) is accomplished using standard in vitro concentrations (0.1-100 nM) of SDF1 siRNAs or siCONTROL Non-targeting siRNA #2 (NTC2) and LipofectAMINE 2000 transfection reagent (Invitrogen, Carlsbad, Calif.). All siRNAs are dissolved in 1×siRNA buffer, an aqueous solution of 20 mM KCl, 6 mM HEPES (pH 7.5), 0.2 mM MgCl₂. Control samples include a buffer control in which the volume of siRNA is replaced with an equal volume of 1×siRNA buffer (Null). SDF1 mRNA level is determined by qRT-PCR using Assays-On-Demand Gene Expression kits, TaqMan Universal PCR Master Mix, and an ABI PRISM 7700 Sequence Detector (Applied Biosystems, Foster City, Calif.). SDF1 mRNA expression is normalized to PPIB (cyclophilin B) mRNA level, and is reported relative to SDF1 expression in non-transfected cells. The SDF1 siRNAs are double-stranded interfering RNAs having specificity for 19-nucleotide sequences contained within the SDF1 mRNA sequence (derived from SEQ ID NO:1, SEQ ID NO:2 and SEQ ID NO:3). It is expected that the siRNAs will cause about at least a 70% reduction in SDF1 expression at 100 mM. Some of the siRNAs may lose efficacy at 10 nM and below. Some of the siRNAs will maintain efficacy at 10 nM, but will lose efficacy at 1 nM and below. Some of the siRNAs will maintain efficacy at 1 nM, and cause significant reduction in SDF1 expression at 0.1 nM.

1. A method of treating a neovascularization-related condition in a subject in need thereof, said method comprising:
administering to an eye of the subject a composition comprising an effective amount of
interfering RNA having a length of 19 to 49 nucleotides, and a pharmaceutically acceptable carrier, wherein
the interfering RNA comprises a region of at least 13 contiguous nucleotides
having at least 90% sequence complementarity to, or at least 90% sequence identity with, the penultimate 13 nucleotides of the 3′ end of an mRNA corresponding to any one of SEQ ID NO:6, SEQ ID NO:75-SEQ ID NO:122, and SEQ ID NO: 170-SEQ ID NO: 213;
wherein the neovascularization-related condition is treated thereby.

2. The method of claim 1, wherein the interfering RNA comprises a region of at least 14, 15, 16, 17, or 18 contiguous nucleotides having at least 85% sequence complementarity to, or at least 80% sequence identity with, the penultimate 14, 15, 16, 17, or 18 nucleotides of the 3′ end of an mRNA corresponding to any one of SEQ ID NO:6, SEQ ID NO:75-SEQ ID NO:122, and SEQ ID NO: 170-SEQ ID NO: 213.

3. The method of claim 1, wherein the composition is administered via an intraocular, or topical route.

4. The method of claim 1, wherein the interfering RNA is administered via in vivo expression from an expression vector capable of expressing the interfering RNA.

5. The method of claim 1, wherein the interfering RNA is an shRNA, an miRNA, or an siRNA.

6. The method of claim 1, wherein the subject is a human and the human has a neovascularization-related condition or is at risk of developing a neovascularization-related condition.

7. A method of attenuating expression of CXCR4 mRNA in an eye of the subject, comprising:
administering to an eye of the subject a composition comprising an effective amount of interfering
RNA having a length of 19 to 49 nucleotides and a pharmaceutically acceptable carrier, the interfering RNA, said method comprising:
a sense nucleotide strand, an antisense nucleotide strand, and a region of at least near-perfect contiguous complementarity of at least 19 nucleotides;
wherein the antisense strand hybridizes under physiological conditions to a portion of
mRNA corresponding to SEQ ID NO:4 beginning at nucleotide 122, 123, 137, 151, 163, 182, 244, 271, 278, 280, 291, 292, 293, 294, 421, 424, 481, 519, 535, 537, 547, 550, 604, 619, 626, 628, 630, 701, 772, 778, 806, 883, 893, 905, 912, 922, 1063, 1070, 1085, 1088, 1091, 1117, 1122, 1123, 345, 459, 486, 521, 522, 525, 600, 639, 647, 832, 867, 925, 962, 963, 979, 1068, 1190, 1191, 1330, 1448, 1557, 1607, 1609, 1610, 1638, 1652, 1658, 1659, 1661, 1662, 1663, 1664, 1732, 1733, 1756, 1757, 1758, 1759, 1764, 159, 164, or 288, or a portion of mRNA corresponding to SEQ ID NO:5 beginning at nucleotide 106; and
wherein the expression of CXCR4 mRNA is attenuated thereby.

8. A method of attenuating expression of SDF1 mRNA in an eye of the subject, comprising:
administering to an eye of the subject a composition comprising an effective amount of interfering
RNA having a length of 19 to 49 nucleotides and a pharmaceutically acceptable carrier, the interfering RNA, said method comprising:
a sense nucleotide strand, an antisense nucleotide strand, and a region of at least near-perfect contiguous complementarity of at least 19 nucleotides;
wherein the antisense strand hybridizes under physiological conditions to a
portion of mRNA corresponding to SEQ ID NO:1 beginning at nucleotide 94, 97, 170, 172, 180, 184, 198, 205, 208, 211, 214, 215, 217, 218, 227, 236, 247, 248, 273, 275, 276, 277, 278, 279, 281, 284, 286, 287, 289, 295, 299, 300, 301, 306, 307, 309, 312, 313, 314, 315, 316, 328, 331, 334, 336, 337, 338, 194, 291, 317, 322, 187, 209, 330, 511, 551, 664, 671, 790, 793, 970, 971, 1267, 1301, 1358, 1359, 1388, 1492, 1493, 1495, 1653, 1701, 1708, 1842, 1857, 1858, 1860, 1913, or 1914, or to a portion of mRNA corresponding to SEQ ID NO:2 beginning at nucleotide 494, 495, 598, 996, 997, 999, 1506, 1695, 1783, 1784, 1792, 1862, 1892, 1893, 2081, 2155, 2249, 2284, 3225, 3386, 3400, 3401, or 3431, or to a portion of mRNA corresponding to SEQ ID NO:3 beginning at nucleotide 447, 449, or 450;
wherein the expression of SDF 1 mRNA is attenuated thereby.

9. The method of claim 7, wherein the subject is a human and the human has a neovascularization-related condition or is at risk of developing a neovascularization-related condition.

10. The method of claim 7, wherein the sense nucleotide strand and the antisense nucleotide strand are connected by a loop nucleotide strand.

11. The method of claim 7, wherein the composition is administered via an intraocular or topical route.

12. The method of claim 7, wherein the interfering RNA is administered via in vivo expression from an expression vector capable of expressing the interfering RNA.

13-22. (canceled)

23. A method of treating a neovascularization-related condition in a subject in need thereof, said method comprising:
administering to an eye of the subject a composition comprising a double stranded siRNA
molecule that down regulates expression of a CXCR4 gene via RNA interference,
wherein:
each strand of the siRNA molecule is independently about 19 to about 27 nucleotides in length; and
one strand of the siRNA molecule comprises a nucleotide sequence having substantial complementarity to an mRNA corresponding to the CXCR4 gene so that the siRNA molecule directs cleavage of the mRNA via RNA interference.

24-28. (canceled)

29. The method of claim 23, wherein each strand of the siRNA molecule is independently about 19 nucleotides to about 25 nucleotides in length or is independently about 19 nucleotides to about 21 nucleotides in length.

30-31. (canceled)
